(19) United States
(12) Patent Application Publication
(10) Pub. No.: US 2021/0060158 A1
(43) Pub. Date: Mar. 4, 2021

(54) AGONIST OF ARYL HYDROCARBON RECEPTOR FOR USE IN CANCER COMBINATION THERAPY

(71) Applicants: INSTITUT CURIE, Paris (FR); INSERM (INSTITUT NATIONAL DE LA SANTE ET DE LA RECHERCHE MEDICALE), Paris (FR)

(72) Inventors: Sebastian Amigorena, Paris (FR); Elodie Segura, Paris (FR)

(21) Appl. No.: 16/644,159

(22) PCT Filed: Sep. 19, 2018
(86) PCT No.: PCT/EP2018/075291

§ 371 Date: Mar. 3, 2020

(30) Foreign Application Priority Data
Sep. 19, 2017 (EP) 17306216.7

(51) Int. Cl. A61K 39/395; A61K 31/404; A61K 31/407; A61K 31/352; A61K 31/353; A61K 31/7048; A61K 31/4439; A61K 31/47; A61K 31/42; A61K 31/505; A61K 31/4184; A61K 31/198; A61K 38/17; A61K 38/44; A61P 35/00

(52) U.S. Cl. CPC A61K 39/3955; A61P 35/00; A61K 31/407; A61K 31/352; A61K 31/353; A61K 31/7048; A61K 31/4439; A61K 31/47; A61K 31/42; A61K 31/505; A61K 31/4184; A61K 31/198; A61K 38/1774; A61K 38/177; A61K 38/44; A61K 38/178; A61K 38/1793; A61K 31/404

(57) ABSTRACT

The present invention relates to an AhR agonist for use in combination with at least one immune checkpoint modulator in the treatment of cancer. The present invention also encompasses product containing an AhR agonist and at least one immune checkpoint modulator as defined in any one of the preceding, claims, as a combined preparation for simultaneous, separate or sequential use in the treatment of cancer.

### FIELD OF THE INVENTION

The present invention relates to the treatment of cancer and in particular to the use of an agonist of the Aryl hydrocarbon receptor (AHR) in combination with immune checkpoint therapy.

### BACKGROUND OF THE INVENTION

Immune checkpoints refer to a plethora of inhibitory and stimulatory pathways hardwired into the immune system that are crucial for maintaining self-tolerance and modulating the duration and amplitude of physiological immune responses in peripheral tissues, in order to minimize collateral tissue damage. Indeed, the balance between inhibitory and stimulatory signals determines the lymphocyte activation and consequently regulates the immune response (Pardoll D M, Nat Rev Cancer. 2012 Mar. 22;12(4):252-64).

It is now clear that tumours co-opt certain immune-checkpoint pathways as a major mechanism of immune resistance, particularly against T cells that are specific for tumour antigens. Because many of the immune checkpoints are initiated by ligand-receptor interactions, they can be readily blocked by antibodies or modulated by recombinant forms of ligands or receptors. Thus agonists of co-stimulatory receptors or antagonists of inhibitory signals, both of which result in the amplification of antigen-specific T cell responses are the primary agent in current clinical testing.

In this context, cancer immunotherapy has been viewed as breakthrough in the field of cancer treatment, switching from targeting the tumor to targeting the immune system (Couzin-Frankel J., Science. 2013 Dec. 20;342(6165):1432-3). The blockade of immune checkpoints with antibodies anti-CTLA-4, PD1 and PD-L1 has given impressive clinical results and manageable safety profiles.

However, only a small proportion of patients respond to these therapies, thus, there is a need to improve cancer immunotherapies by new approaches and/or by combining anti-checkpoint antibodies with other treatments. Moreover, anti-checkpoint antibodies can induce side effects, mainly autoimmunity, such that implementing combination therapies which may help lower the administered doses, and consequently the adverse events, remains of invaluable medical help.

In the same time, among the innate and adaptive immune cells recruited to the tumor site, macrophages are particularly abundant and are present at all stages of tumor progression. Clinical and experimental results have shown that these macrophages generally play a protumoral role. In the primary tumor, macrophages can stimulate angiogenesis and enhance tumor cell invasion, motility, and intravasation. During monocytes and/or metastasis, macrophages prime the premetastatic site and promote tumor cell extravasation, survival, and persistent growth. Macrophages are also immunosuppressive, notably by preventing tumor cell attack by natural killer and T cells during tumor progression and after recovery from chemo- or immunotherapy. (see notably Roy Noy & Jeffrey W. Pollard, Immunity 41, Jul. 17, 2014). Radiotherapy and chemotherapy are useful treatments in many cancers, and studies have shown that infiltrated-myeloid increases after irradiation. However, the interaction between tumor cells and stroma after these therapies remains poorly defined. DNA damage, cell death, and increased hypoxia have been observed in tumors after radiotherapy, which has been shown to lead to macrophage recruitment and promote tumor progression in animal models. It has therefore been proposed to combine macrophages targeting therapies with standard therapies such as radiotherapy and chemotherapy (Yang L, Zhang Y. Tumor-associated macrophages, potential targets for cancer treatment. Biomarker Research. 2017;5:25. doi:10.1186/s40364-017-0106-7).

Thus there remains a need for implementing combination therapies; in particular there remain a need for improving the efficacy of cancer immunotherapies with limiting adverse side effects.

### SUMMARY OF THE INVENTION

The inventors have now discovered that AHR silencing reduced monocyte-derived dendritic (mo-DC) differentiation while slightly increasing monocyte-derived macrophage (mo-Mac) differentiation. They further demonstrated that AHR activation increased mo-DC while decreasing mo-Mac development both in vitro and in vivo. To the inventor's knowledge these results show for the first time that AHR is a molecular switch for mo-DC vs. mo-Mac differentiation.

Furthermore, while AHR had been shown to be chronically activated in tumours, thus facilitating tumour progression (see notably Murray I. A et al, Nature Review Cancer, vol. 14, 2014). The inventors also provide results that demonstrate also for the first time that the anti-tumor effect of an immune checkpoint modulator treatment is greatly enhanced when combined with an AHR agonist treatment. In particular, they showed that surprisingly, while anti-PD1 treatment had only moderate effect on mice survival after tumor inoculation, and while a treatment with an AHR agonist, notably a food supplied AHR agonist, did not improved survival, their combination leads to a drastic and sustained improvement of survival.

Thus the present invention relates to an AHR agonist for use in combination with at least one immune checkpoint modulator in the treatment of cancer.

### Definitions

The abbreviations “AHR” and “AhR” are equivalent and designate both the Aryl hydrocarbon receptor.

“Treatment”, or “treating” as used herein, is defined as the application or administration of a therapeutic agent or combination of therapeutic agents (e.g., an agonist of AHR and/or an immune checkpoint modulator) to a patient, or application or administration of said therapeutic agents to an isolated tissue or cell line from a patient, who has a cancer with the purpose to cure, heal, alleviate, relieve, alter, remedy, ameliorate, improve or affect the cancer, or any symptom of the cancer. In particular, the terms “treat' or treatment” refers to reducing or alleviating at least one adverse clinical symptom associated with cancer, e.g., pain, swelling, low blood count etc.

In another embodiment, the term “treat' or treatment” refers to slowing or reversing the progression neoplastic uncontrolled cell multiplication, i.e. shrinking existing tumors and/or halting tumor growth.

The term “treat' or treatment” also refers to inducing apoptosis in cancer or tumor cells in the subject.

The term “treatment” or “treating” is also used herein in the context of administering the therapeutic agents prophylactically.

The term “effective dose” or “effective dosage” is defined as an amount sufficient to achieve, or at least partially achieve, the desired effect. The term “therapeutically effective dose” is defined as an amount sufficient to cure or at least partially arrest the disease and its complications in a patient already suffering from the disease. The term “patient” includes human and other mammalian subjects that receive either prophylactic or therapeutic treatment.

As used herein, the term “therapeutically effective regimen” refers to a regimen for dosing, timing, frequency, and duration of the administration of one or more therapies according to the invention (i.e., the AHR agonist and the at least one immune checkpoint modulator), for the treatment and/or the management of cancer or a symptom thereof. In a specific embodiment, the regimen achieves one, two, three, or more of the following results: (1) a stabilization, reduction or elimination in the cancer cell population; (2) a stabilization or reduction in the growth of a tumor or neoplasm; (3) an impairment in the formation of a tumor; (4) eradication, removal, or control of primary, regional and/or metastatic cancer; (5) a reduction in mortality; (6) an increase in disease-free, relapse-free, progression-free, and/or overall survival, duration, or rate; (7) an increase in the response rate, the durability of response, or number of patients who respond or are in remission; (8) a decrease in hospitalization rate, (9) a decrease in hospitalization lengths, (10) the size of the tumor is maintained and does not increase or increases by less than 10%, preferably less than 5%, preferably less than 4%, preferably less than 2%, and (11) an increase in the number of patients in remission.

As used herein, the term “in combination”, or “combined administration” in the context of the invention refers to the administration of an AHR agonist and of at least one immune checkpoint modulator to a patient for cancer therapeutic benefit. The term “in combination” in the context of the administration can also refer to the prophylactic use of an AHR agonist when used with at least one immune checkpoint modulator.

The use of the term “in combination” does not restrict the order in which the therapies (e.g., the AHR agonist and the at least one immune checkpoint modulator) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a patient which had, has, or is susceptible to cancer. The therapies are administered to a patient in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The results of the present invention have established a basis for dual treatment of patients with an AHR agonist and at least one immune checkpoint modulator such as an anti-PD-1 antibody, and/or an anti-PD1 antibody and/or an anti-CTLA-4 antibody. These two therapies need not be given concurrently, but can also be given sequentially and/or on different duration. Typically, the patient may be chronically supplied with an AHR agonist, notably in the form of a medical food composition, while sequence treatment with the immune checkpoint modulator may be administered. Accordingly, and as used herein, the expression “An agonist of AHR for use in combination with at least one immune checkpoint modulator in the treatment of cancer” can be used interchangeably with the expression “At least one immune checkpoint modulator for use in combination with an AHR agonist in the treatment of cancer”.

The terms “synergy,” “synergistic,” or “synergistic effect” as used herein describe an effect that has a magnitude that is greater than the sum if the individual effects. In some embodiments of the present invention, the use of both an AHR agonist and an immune checkpoint modulator in concert provides a synergistic therapeutic effect on a neoplastic condition in a patient and/or on the growth of a cell.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab')2 fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of kappa or lambda types. In one embodiment, the antibody is glycosylated. An antibody can be functional for antibody-dependent cytotoxicity and/or complement-mediated cytotoxicity, or may be non-functional for one or both of these activities.

A measure of the affinity of a ligand for its receptor can be the equilibrium dissociation constant K_{D}. The affinity thus defines the relation between concentration of a ligand and fractional occupancy of the receptor and it is usually the highest for hormones and natural ligands. Alternatively, a measure of affinity could be expressed as the half maximal effective concentration EC₅₀ that corresponds to an ability of a drug to elicit 50% of maximal effect. The ability of drug to elicit measurable functional change is called potency. Relative maximal effect of a drug in certain tissue in comparison with Natural (or another highly potent) ligand is called intrinsic activity (IA). According to intrinsic activity, several types of ligands can be distinguished, including full agonists (IA=1, i.e. having 100% intrinsic activity), partial agonists (IA=0˜1), antagonists (IA=0) and inverse agonist (IA<0).

In the context of this the invention, a “weak agonist” refers to an aryl hydrocarbon receptor ligand that displays partial agonist activity, eliciting a sub-maximal dioxin-responsive element-mediated transcriptional response. A “full agonist” means an aryl hydrocarbon receptor ligand that maximally elicits canonical dioxin-responsive element-mediated transcriptional responses.

As per the invention, “endogenous compounds” are compounds that are also endogenously synthetized as opposed to purely synthetic compounds. “Exogenous compounds” may be synthetic or natural compounds.

### DETAILED DESCRIPTION OF THE INVENTION

The present invention relates to an AHR agonist for use in combination with at least one immune checkpoint modulator in the treatment of cancer.

### Agonists of AHR

As used herein the term AHR has its usual meaning in the art and refer to the Aryl Hydrocarbon receptor also called the dioxin receptor which is a ligand activated transcriptional factor of the basic helix-loop-leix/per-ARNT-Sim (bHLH/PAS) superfamily. It is widely expressed in the body and evolutionarily conserved from invertebrates onwards but its activity is tightly controlled.

In addition to the regulation of the CYP1 family of xenobiotic metabolizing enzymes by AHR via exogenous ligands, recent recognition of endogenous AHR ligands provided evidence that AHR also plays a role in many physiological functions such as notably the regulation of the cell cycle and proliferation, immune response, circadian rhythm, tumor promotion and the expression of lipid metabolism genes. The AhR gene is about 50 kb long and contains 11 exons. The AhR gene encodes a 96 kDa protein (see Bennett, P.; Ramsden, D. B.; Williams, A. C. Complete structural characterisation of the human aryl hydrocarbon receptor gene. Clin. Mol. Pathol., 1996, 49(1), M12-M16; and Dolwick, K. M.; Schmidt, J. V.; Carver, L. A.; Swanson, H. I.; Bradfield, C. A. Cloning and expression of a human Ah receptor cDNA. Mol. Pharmacol., 1993, 44(5), 911-7). The human AHR is referenced P35869 (AHR_HUMAN) in UNIPROT.

In its resting state, AhR is sequestered in the cytosol in a multiprotein complex with heat shock protein 90 (hsp90) and other proteins. Hydrophobic ligands penetrate through the cell membrane and bind to AhR, which in turn undergoes cytosol to nucleus translocation. In the nucleus, AhR forms a heterodimer with AhR nuclear translocator (ARNT). AhR/ARNT complex then binds to specific DNA sequence called dioxin responsive element (DRE) or xenobiotic responsive element (XRE) in the promoter of target genes and triggers their expression. (see for review: Lucie Stejskalova, Zdenek Dvorak and Petr Pavek; Endogenous and Exogenous Ligands of Aryl Hydrocarbon Receptor: Current State of Art, Current Drug Metabolism, 2011, 12, 198-212).

Examination of an agonistic AHR activity as per the invention may for example be achieved by following the capacity of a given compound to activate. The capacity of a compound to influence AHR-mediated gene expression can be examined for example in mouse model cells (notably Hepa1.1 or HepG2 cells) stably harboring an AHR responsive luciferase reporter construct. The cells can be incubated with vehicle, 10 nM TCDD or increasing concentrations of the tested compound. Increase in the reporter activity means that the compound as activates AHR. Said activation may be compared with TCDD activation for reference and identification of partial or full agonist activity (for detailed protocol see for example Hubbard T D, Murray I A, Bisson W H, et al. Adaptation of the human aryl hydrocarbon receptor to sense microbiota-derived indoles. Scientific Reports. 2015;5:12689).

The majority of AHR ligands are partial or weak agonists. While partial agonists can have similar affinity as full agonists the intrinsic activity of a partial agonist is lower than that of a full agonist. Most compounds that bind and activate AHR are also hydrophobic molecules. AHR agonists are notably described in Joshua D. Mezrich et al. “An Interaction between Kynurenine and the Aryl Hydrocarbon Receptor Can Generate Regulatory T Cells” J Immunol 2010; 185:3190-3198; Mulero-Navarro S and Fernandez-Salguero P M (2016) New Trends in Aryl Hydrocarbon Receptor Biology. Front. Cell Dev. Biol. 4:45; Lucie Stejskalova et al. Endogenous and Exogenous Ligands of Aryl Hydrocarbon Receptor: Current State of Art, Current Drug Metabolism, 2011, 12, 198-212; Brigitta Stockinger et al. The Aryl Hydrocarbon Receptor: Multitasking in the Immune system Annu. Rev. Immunol. 2014. 32:403-32.

As above mentioned, the inventors have discovered and demonstrated that the AHR activation leads to monocyte-derived dendritic (mo-DC) differentiation while decreasing monocyte-derived Macrophage (mo-Mac) development, such that AHR activation acts as a molecular switch for monocyte fate specification. Therefore they proposed to combine immune checkpoint treatment with therapy aimed to activate the AHR receptor for the treatment of cancer. Thus preferably, the AHR ligand, in particular the AHR agonist as per the invention does not lead to mo-Mac differentiation. Estimation of the impact on mo-DC vs mo-Mac differentiation of a selected AHR agonist as previously listed can be achieved as illustrated in the results mentioned in the examples (see notably FIG. 1). In particular, mo-DC vs mo-Mac differentiation may be assessed on in vitro monocytes cultivated in the presence of various doses of the selected agonist. Expression of the mo-DC vs the mo-Mac signature can be assessed by RTqPCR. Typically expression of IRF4 is indicative of a mo-DC signature.

Surprisingly the inventors have found that administration of the AHR agonist, according to the present invention, (notably the dietary AHR agonist I3C) does not induce functional T_{reg} cells. Thus preferably also the selected AHR agonist as per the invention does not induce functional T_{reg} cell conversion upon administration. The impact of a given AHR agonist on T_{reg} cells may be estimated by exposing purified T cells (typically CD4⁺ T cells) in vitro with the said agonist. T_{reg} cells are characterized by expression of the transcription factor Foxp3. Thus detection of Foxp3 expression for example through RTqPCR is indicative of T_{reg} cell conversion (see notably for details Quintana F J et al., Control of Treg and Th17 cell differentiation by the aryl hydrocarbon receptor, Nature 2008,453(1):65-71, and Feuerer Markus et al. Genomic definition of multiple ex vivo regulatory T cell subphenotypes. PNAS. 2010;107(13):5919-5924).

Thus preferably the invention relates to an AHR agonist (notably one or more AHR agonist) as listed in the present application, for use in combination with at least one immune checkpoint modulator in the treatment of cancer;

wherein said AHR agonist does not lead to mo-Mac differentiation and does not induce functional T_{reg} cell conversion.

Preferably according to the invention, the AHR agonist is selected from the group comprising dietary indoles, dietary flavonoids, tryptophan metabolites and synthetic weak AHR agonists.

### Indolyl Derivatives

In one embodiment, the AHR agonist is an indolyl derivative (also named indole derivatives) such as indolyl compounds generated by the tryptophan metabolism and/or derived from dietary intake compounds.

### a) Indolyl Derivatives from the Tryptophan Metabolism

The AHR agonist candidates can be derived from the tryptophan metabolism, notably tryptophan indolyl metabolites. Indole is the functional group that defines the amino acid tryptophan and is a chemical component of the neurotransmitter 5-hydroxytryptamine, the hormone melatonin, and the plant signaling and pigment molecules auxin and indigo, respectively. Tryptophan is an essential amino acid and a precursor of many vital components in the body. Several degradation pathways generate tryptophan metabolites with AHR-inducing activity, which are encompassed in the present invention as AHR agonists. Preferably those compounds are endogenous compounds.

The major pathway of tryptophan metabolism in the body proceeds via the enzymes indoleamine 2,3-dioxygenase (IDO) and tryptophan 2,3-dioxygenase (TDO), which generate the metabolite kynurenine and its derivative thereof or downstream metabolites such as kynurenic acid and xanthurenic acid.

Tryptophan metabolites according to the present invention also encompass metabolites catalyzed by tryptophan hydroxylase and dopamine decarboxylase such as tryptamine (TA), and indole acetic acid (IAA); compounds from the serotonin pathway such as hydroxytryptamine, or 5-hydroxytryptamine, 5-Hydroxytryptophan; and 6-formylindolo[3,2-b]carbazole (FICZ) which is notably produced by exposure of L-tryptophan to UVB radiation.

### b) AHR Agonists Derived from the Tryptophan Bacterial Metabolism

AHR agonists from the tryptophan metabolism as per the invention also include metabolites from the bacterial metabolism and most particularly, metabolites from the commensal bacterial metabolism. Indeed, commensal bacteria expressing tryptophanase catabolize tryptophan to indole, a quorum-sensing compound for bacteria. For example, Lactobacillus spp. converts tryptophan to indole-3-aldehyde (I3A) and Clostridium sporogenes convert tryptophan to IPA, likely via a tryptophan deaminase. After absorption from the intestinal tract into portal circulation, the liver converts indole to Indol Sulfate (see also Zhang L S, Davies S S. Microbial metabolism of dietary components to bioactive metabolites: opportunities for new therapeutic interventions. Genome Medicine. 2016;8:46.). AHR agonists from the tryptophan bacterial metabolism include indoxyl sulfate, indole-3-acetic acid (IAA or indole acetate), indole-3-pyruvic acid (IPA, or indole pyruvate), indole-3-carbinol (I3C, or indole carbinol), indole-3-aldehyde (or indole aldehyde), tryptamine, 3-methylindole, indirubin and malassezin.

Thus an AHR agonist from the tryptophan metabolism as per the invention can be selected from the group comprising kynurenine, kynurenic acid, xanthurenic acid, tryptamine (TA), indole acetic acid (IAA); compounds from the serotonin pathway such as hydroxytryptamine, or 5-hydroxytryptamine, 5-Hydroxytryptophan; 6-formylindolo[3,2-b]carbazole (FICZ); metabolites from the commensal bacterial metabolism such as indoxyl sulfate, indole-3-acetic acid (IAA or indole acetate), indole-3-pyruvic acid (IPA, or indole pyruvate), indole-3-carbinol (I3C, or indole carbinol), indole-3-aldehyde (or indole aldehyde), tryptamine, 3-methylindole, indirubin and malassezin. Preferably, an AHR agonist from the tryptophan metabolism is selected from kynurenine, kynurenic acid, FICZ, IAA, IPA, I3C and idoxyl sulfate, most preferably the AHR agonist from the tryptophan metabolism is FICZ or I3C.

### c) Dietary Indolyl AHR Agonists

Indolyl AHR agonists can also be dietary AHR ligands (dietary indols) including indol glucosinolate such as (3-indolylmethyl glucosinolate, also named glucobrassicin) and indole-3-carbinol (I3C) (which is an autolysis compound of 3-indolylmethyl glucosinolate) and their derivatives notably the derivatives which are generated by the metabolism of dietary intake such as, 3,3′-diindoylmethane (DIM) and Indolo[3,2b]carbazole (ICZ) (see also for reference Bjeldanes, L. F., Kim, J. Y., Grose, K. R., Bartholomew, J. C., and Bradfield, C. A. (1991) Aromatic hydrocarbon responsiveness-receptor agonists generated from indole-3-carbinol in vitro and in vivo: comparisons with 2,3,7,8-tetrachlorodibenzo-p-dioxin. Proc Natl Acad Sci U S A 88, 9543). Indole glucosinolate occurs naturally in a large amount in a number of vegetables of the Brassica genus, such as cruciferous vegetables, cabbages, or mustard plants including but not limited to the root (rutabaga, turnip), stems (kohlrabi), leaves (cabbage, collard greens, kale), flowers (cauliflower, broccoli), buds (Brussels sprouts, cabbage), and seeds (many, including mustard seed, and oil-producing rapeseed).

### Other Dietary AHR Agonist

In one embodiment, an AHR agonist as per the invention can also be selected from dietary flavonoids. Flavonoids include flavones, flavonols, flavanones, isoflavones and cathechins. Preferably, a flavonoid AHR agonist is selected from quercetin, galangin, daidzein, resveratrol, naringenin, baicalein diosmin and diametin. Most preferably a flavonoid AHR agonist is selected from quercetin, galangin and naringenin.

Thus a dietary AHR agonist as per the invention can be selected from the group comprising dietary indoles and dietary flavonoids as defined above.

Typically a dietary AHR agonist is in the form of a natural product extract. The natural product extract may be of vegetal (including fungal and algae) or animal origin. Preferably the natural extract is a vegetal. A vegetal extract according to the present invention include the full vegetal but also in a non-limitative way roots, rhizomes, wood, barks, leaves, flowers, flower buds, fruits, seeds, fruit juices, or plant excretions (gums or exudates) as well as any crude or refined preparations obtained from such extracts (such as but not limited to infusion, decoction, alcoholic tincture, juice, oleoresin, essential oil).

### Synthetic AHR Agonists

In one embodiment of the invention, the AHR agonist is selected from synthetic compounds. In particular, an AHR agonist can be selected from benzimidazole derivatives such as omeprazole and lansoprazole; A synthetic AHR agonist as per the invention can also be selected from the group comprising primaquine, leflutamide, VAF347 ([4-(3-chloro-phenyl)-pyrimid in-2-yl]-(4trifluoromethyl-phenyl)-amine), TSU-16 ((Z)-3-[(2,4-dimethylpyrrol-5-yl)methylidenyl]-2indolinone), synthetic flavonoids such as TMF (6,2′,4′-trimethoxyflavone) and MNF (3′-methoxy-4′nitroflavone), M50367 (ethyl 3-hydroxy-3-[2-(2-phenylethyl)benzoimidazol-4-yl]propanoate), and M50354 (3-[2-(2-phenylethyl)benzoimidazole-4-yl]-3-hydroxypropanoic acid). Preferably, the synthetic AHR agonist of the invention is a weak AHR agonist.

In a preferred embodiment, the AHR agonist is I3C or FICZ.

### Composition Comprising the AHR Agonist

In one embodiment of the present invention the AHR agonist is in the form of a composition, preferably the composition is suitable for oral or enteral administration. Most preferably the composition is suitable for oral administration.

Preferably also the composition is in the form of a medical food or a dietary supplement. The composition typically comprises one or more AHR agonist(s) as previously described.

A medical food is a food which is formulated to be consumed or administered enterally under the supervision of a physician and which is intended for the specific dietary management of a disease or condition for which distinctive nutritional requirements, based on recognized scientific principles, are established by medical evaluation.

Dietary supplement are products intended to supplement the diet. They can be in a large variety of forms such as traditional tablets, capsules, and powders, as well as drinks and energy bars.

Preferably the medical food composition, or dietary supplement comprises, one or more dietary AHR agonist(s) such as a dietary indoles or dietary flavonoids as described above. Most preferably the composition comprises at least indole glucosinolate such as (3-indolylmethyl glucosinolate, also named glucobrassicin) and/or indole-3-carbinol (I3C). In a preferred embodiment, indole glucosinolate or I3C is in the form of a natural crude or refined extract of one or more vegetables of the Brassica genus.

In one embodiment, the composition and notably the medical food composition or the dietary supplement comprises prebiotic and/or probiotic compounds promoting development of commensal bacteria producing AHR agonists, and notably producing bacterial tryptophan metabolites as previously defined. In one embodiment, the composition can comprise one or more AHR agonist, notably one or more dietary agonist, and one or more prebiotic and/or probiotic compounds as mentioned above.

### Immune Checkpoint Modulators

As used herein the term “immune checkpoint protein” has its general meaning in the art and refers to a molecule that is expressed by T cells and/or by NK cells and that either turn up a signal (stimulatory checkpoint molecules) or turn down a signal (inhibitory checkpoint molecules). Most preferably according to the invention the immune checkpoint molecule is at least expressed by T cells.

Immune checkpoint molecules are recognized in the art to constitute immune checkpoint pathways similar to the CTLA-4 and PD-1 dependent pathways. Immune checkpoint molecules according to the invention are notably described in Pardoll, 2012. Nature Rev Cancer 12:252-264; Mellman et al., 2011. Nature 480:480-489; Chen L & Flies D B, Nat. Rev. Immunol. 2013 April; 13(4):227-242, and Kemal Catakovic, Eckhard Klieser et al., “T cell exhaustion: from pathophysiological basics to tumor immunotherapy” Cell Communication and Signaling 2017,15:1). Example of immune checkpoints molecules notably encompasses CD27, CD40, OX40, GITR, ICOS, TNFRSF25, 41BB, HVEM, CD28, TMIGD2, CD226, 2B4 (CD244) and ligand CD48, B7-H6 Brandt (NK ligand), LIGHT (CD258, TNFSF14), CD28H, A2AR, B7-H3, B7-H4, BTLA, CTLA-4, CD277, IDO, KIRs, PD-1s, LAG-3, TIM-3 TIGIT, VISTA, CD96, CD112R, CD160, CD244 (or 2B4) DCIR (C-type lectin surface receptor), ILT3, ILT4 (Immunoglobulin-like transcript), CD31 (PECAM-1) (Ig-like R family), CD39, CD73, CD94/NKG2, GP49b (immunoglobulin superfamily), KLRG1, LAIR-1 (Leukocyte-associated immunoglobulin-like receptor 1) CD305 , PD-L1 and PD-L2 and SIRPα.

Non-limitative examples of inhibitory checkpoint molecules include A2AR, B7-H3, B7-H4, BTLA, CTLA-4, CD277, IDO, KIRs, PD-1, LAG-3, TIM-3 TIGIT, VISTA, CD96, CD112R, CD160, DCIR (C-type lectin surface receptor), ILT3, ILT4 (Immunoglobulin-like transcript), CD31 (PECAM-1) (Ig-like R family), CD39, CD73, CD94/NKG2, GP49b (immunoglobulin superfamily), KLRG1, LAIR-1 (Leukocyte-associated immunoglobulin-like receptor 1), CD305, PD-L1 and PD-L2.

The Adenosine A2a receptor (A2aR), the ligand of which is adenosine, is regarded as an important checkpoint in cancer therapy because adenosine in the immune microenvironment, leading to the activation of the A2a receptor, is negative immune feedback loop and the tumor microenvironment has relatively high concentrations of adenosine. A2aR can be inhibited by antibodies that block adenosine binding or by adenosine analogues some of which are fairly specific for A2aR. These drugs have been used in clinical trials for Parkinson's disease.

The B7 family is an important family of membrane-bound ligand that binds co-stimulatory and inhibitory receptors. All of the B7 family members and their known ligands belong to the immunoglobulin superfamily. Many receptors have not been yet identified. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape.

CD160 is a glycosylphosphatidylinositol (GPI)-anchored protein member of the Ig superfamily with a restricted expression profile that is limited to CD56dim CD16+ NK cells, NKT-cells, yδ T-cells, cytotoxic CD8+ T-cells lacking the expression of CD28, a small fraction of CD4+ T cells and all intraepithelial lymphocytes. Binding of CD160 to both classical and non-classical MHC I enhances NK and CD8+ CTL functions. However, engagement of CD160 by the Herpes Virus Entry Mediator (HVEM/TNFRSF14) was shown to mediate inhibition of CD4+ T-cell proliferation and TCR-mediated signaling.

HVEM (Herpesvirus Entry Mediator) protein is a bimolecular switch that binds both co-stimulatory LT-α/LIGHT and co-inhibitory receptors BTLA/CD160. The ligation of coinhibitory receptors BTLA and/or CD160 on T cells with HVEM expressed on DC or Tregs transduces negative signals into T cells that are counterbalanced by costimulatory signals delivered after direct engagement of HVEM on T cells by LIGHT expressed on DC or more likely, on other activated T cells (T-T cell cooperation). The predominance of the interaction of HVEM with BTLA and CD160 over the HVEM/LIGHT pathway or vice versa might be the result of differences in ligand/receptor affinity and the differential expression pattern of these molecules on cell types at different stages of cell differentiation. LIGHT, BTLA, and CD160 have substantially different binding affinities and occupy spatially distinct sites upon interaction with the HVEM receptor, which enables HVEM to function as a molecular switch. The net effect of the LIGHT/HVEM and HVEM/BTLA/CD160 interaction, when these different receptors and ligands are simultaneously present, determines the outcome of the response (see M. L. del Rio. “HVEM/LIGHT/BTLA/CD160 cosignaling pathways as targets for immune regulation” Journal of Leukocyte Biology. 2010; 87).

B and T Lymphocyte Attenuator (BTLA), also called CD272, has also HVEM as its ligand. BTLA T cells are inhibited in the presence of its ligand, HVEM. Surface expression of BTLA is gradually downregulated during differentiation of human CD8+ T cells from the naive to effector cell phenotype, however tumor-specific human CD8+ T cells express high levels of BTLA (Kenneth M. Murphy et al. Balancing co-stimulation and inhibition with BTLA and HVEM. Nature Reviews Immunology 2006, 6, 671-681).

CTLA-4, Cytotoxic T-Lymphocyte-Associated protein 4 also called CD152, was the first immune checkpoint to be clinically targeted. It is expressed exclusively on T cells. It has been proposed that its expression on the surface of T cells dampens the activation of T cells by outcompeting CD28 in binding CD80 and CD86 as well as actively delivering inhibitory signals to the T cells. Expression of CTLA-4 on T_{reg} cells serves to control T cell proliferation. Ig-like transcript-3 and -4 (ILT3 and ILT4) are inhibitory receptors both expressed by monocytes, macrophages, and DCs. The corresponding ILT3 ligand is not yet known, but since ILT3 can directly suppress T lymphocyte function, it is likely to be expressed on T cells. In several cancers, ILT3 has been found to mediate the immune escape mechanism by impairing T cell responses. Furthermore, ILT4-expressing DCs block efficient CTL differentiation, a mechanism that is used by tumors, which upregulate ILT4 to evade the immune system (Vasaturo A et al., Front Immunol. 2013; 4: 417).

Platelet endothelial cell adhesion molecule-1 (PECAM-1), also known as CD31, is a type I transmembrane glycoprotein member of the immunoglobulin (Ig) gene superfamily which contains six extracellular Ig domains and two cytoplasmic immunoreceptor tyrosine-based inhibitory motifs (ITIMs). PECAM-1 is restricted to endothelial cells and cells of the hematopoietic system (see Newman D K, Fu G, Adams T, et al. The adhesion molecule PECAM-1 enhances the TGFβ-mediated inhibition of T cell function. Science signaling. 2016;9(418):ra27).

LAIR-1 is expressed in very high and relatively homogenous levels in naive T cells but in lower and more heterogeneous levels in memory T cells. LAIR-1 consist of a type I transmembrane glycoprotein of 287 amino acids with a single extracellular C2-type Iglike domain and a cytoplasmic domain with two ITIM motifs. LAIR-1 can inhibit TCR mediated signals possibly through the recruitment of C-terminal Csk, one or more of the phosphatases SHIP, SHP-1 or SHP-2, and to a certain extent on signalling through p38 MAP kinase and ERK signaling (Thaventhiran T et al. (2012) J Clin Cell Immunol S12:004).

IDO1, Indoleamine 2,3-dioxygenase 1, is a tryptophan catabolic enzyme. A related immune-inhibitory enzymes. Another important molecule is TDO, tryptophan 2,3-dioxygenase. IDO1 is known to suppress T and NK cells, generate and activate Tregs and myeloid-derived suppressor cells, and promote tumour angiogenesis.

KIR, Killer-cell Immunoglobulin-like Receptor, are a braid category of inhibitory receptors that can be divided into two classes based on structure: killer cell immunoglobulin-like receptors (KIRs) and C-type lectin receptors which are type II transmembrane receptors. There receptors where originally described as regulators of the killing activity of NK cells although many are expressed on T cells and APCs. Many of the KIRs are specific for subsets MHC class I molecules and possess allele-specificity.

LAG3, Lymphocyte Activation Gene-3 has, as its ligand, MHC class II molecules, which are upregulated on some epithelial cancers but are also expressed on tumour-infiltrating macrophages and dendritic cells. This immune checkpoint works to suppress an immune response by action to T_{reg} cells as well as direct effects on CD8+ T cells.

PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment.

TIM-3 short for T-cell Immunoglobulin domain and Mucin domain 3 (also named B7H5), and the ligand of which is galacting 9, is expressed on activated human CD4+ T cells and regulates Th1 and Th17 cytokines. TIM-3 acts as a negative regulator of Th1/Tc1 function by triggering cell death upon interaction with its ligand, galectin-9.

VISTA (short for V-domain Ig suppressor of T cell activation) VISTA, also known as c10orf54, PD-1H, DD1α, Gi24, Dies1, and SISP1] is a member of the B7 family of NCRs and represents a new target for immunotherapy. Murine VISTA is a type I transmembrane protein with a single IgV domain with sequence homology to its B7 relatives with conserved segments thought to be critical for the IgV stability. VISTA is expressed on naïve T cells whereas PD-1 and CTLA-4 are not, which may suggest that VISTA functions to restrain T cell activity at an even earlier stage in T cell priming. VISTA is expressed on both T cells and APCs with very high expression on myeloid cells. VISTA is hematopoietically restricted and in multiple cancer models, VISTA was only detected on tumor infiltrating leukocytes and not on tumor cells. This unique surface expression pattern suggests that VISTA may function to restrict T cell immunity at different stages. VISTA has been demonstrated to exert both ligand and receptor functions. First, VISTA can function as a ligand to negatively regulate T cell activation. Second, VISTA has been demonstrated to function as a receptor on T cells which negatively regulates their activity. VISTA^{-/-} CD4⁺ T cells respond more vigorously than wild type (WT) CD4⁺ T cells to both polyclonal and antigen specific stimulation leading to increased proliferation and production of IFNγ, TNFα, and IL-17A. Anti-VISTA monotherapy reduced tumor growth in multiple pre-clinical models, B16OVA melanoma, B16-BL6 melanoma, MB49 bladder carcinoma, and PTEN/BRAF inducible melanoma (see Deng J, Le Mercier I, Kuta A, Noelle R J. “A New VISTA on combination therapy for negative checkpoint regulator blockade. J Immunother Cancer. 2016 Dec. 20;4:86. doi: 10.1186/s40425-016-0190-5. eCollection 2016. Review; see also Kathleen M. Mahoney et al., “Combination cancer immunotherapy and new immunomodulatory targets”. Nature Reviews Drug Discovery 2015; 14:561-584).

CD96, CD226 (DNAM-1) and TIGIT belong to an emerging family of receptors that interact with nectin and nectin-like proteins. CD226 activates natural killer (NK) cell-mediated cytotoxicity, whereas TIGIT reportedly counterbalances CD226.

CD96 competes with CD226 for CD155 binding and limits NK cell function by direct inhibition (Christopher J Chan et al., “The receptors CD96 and CD226 oppose each other in the regulation of natural killer cell functions”, Nature Immunology 2014 15, 431-438).

TIGIT (also called T cell immunoreceptor with Ig and ITIM domains, or VSTM3) TIGIT/VSTM3 is expressed normally by activated T cells, regulatory T (T_{reg}) cells, and natural killer (NK) cells. The poliovirus receptor (CD155/PVR) and Nectin-2 (CD112) as well as CD 113 have been identified as relevant ligands. TIGIT/VSTM3 competes with the molecules CD226 and CD96 for binding to CD155/PVR and CD112, respectively, but among all respective receptor-ligand combinations, TIGIT/VSTM3 exhibits the strongest affinity for CD155/PVR. TIGIT inhibits T cell activation in vivo (see Karsten Mahnke et al. TIGIT-CD155 Interactions in Melanoma: A Novel Co-Inhibitory Pathway with Potential for Clinical Intervention. Journal of Investigative Dermatology. 2016; 136: 9-11).

CD112R (PVRIG), the ligand of which is PVRL2, is a member of poliovirus receptor-like proteins which is preferentially expressed on T cells and inhibits T cell receptor-mediated signals.

Non-limitative examples of stimulatory checkpoint molecules include CD27, CD40L, OX40, GITR, ICOS, TNFRSF25, 41BB, HVEM, CD28, TMIGD2, and CD226, 2B4 (CD244) and its ligand CD48, B7-H6 Brandt (NK ligand), CD28H and LIGHT (CD258, TNFSF14).

CD27, CD40L, OX40, GITR, ICOS, HVEM, 2B4 (CD244) and its ligand CD48, B7-H6 Brandt (NK ligand), LIGHT (CD258, TNFSF14), CD28H and TNFSF25 are stimulatory checkpoint molecules, which are members of the tumor necrosis factor (TNF) receptor superfamily (TNFSF). TNFRSF proteins play an important role in B and T cell development, survival, and antitumor immune response. In addition, some TNFRSFs are involved in the deactivation of T_{reg} cells. Therefore, TNFRSF agonists activate tumor immunity, and their combination with immune checkpoint therapy is promising. Several antibodies that act as TNFRSF agonist have been evaluated in clinical trials (Shiro Kimbara and Shunsuke Kondo, “Immune checkpoint and inflammation as therapeutic targets in pancreatic carcinoma”, World J Gastroenterol. 2016 Sep. 7; 22(33): 7440-7452, see also for review Watts TH. TNF/TNFR family members in costimulation of T cell responses. Annu Rev Immunol. 2005; 23:23-68.).

CD27 supports antigen-specific expansion of naïve T cells and is vital for the generation of T cell memory. CD27 is also a memory marker of B cells. CD27's activity is governed by the transient availability of its ligand, CD70, on lymphocytes and dendritic cells. CD27 costimulation is known to suppress Th17 effector cell function.

The CD40:CD40L pathway is a co-stimulatory pathway that affects both humoral and cell-mediated immunity. CD40L (also known as CD154), is primarily expressed on T-helper cells shortly after activation. The receptor 2B4 (CD244) belongs to the signaling lymphocyte activation molecule (SLAM) subfamily within the immunoglobulin superfamily (IgSV). All members of this family contain two or more immunoreceptor tyrosine-based switch motifs (ITSMs) in their cytoplasmatic tail including the receptors CD229, CS1, NTB-A and CD84 [92]. 2B4 is expressed by NK cells, yδ T cells basophils and monocytes, upon activation on CD8+ T cells and binds with high affinity to CD48 on lymphoid and myeloid cells (Kemal Catakovic et al., Cell Communication and Signaling201715:1).

TNFSF14/LIGHT/CD258 exhibits inducible expression, and competes with herpes simplex virus (HSV) glycoprotein D for herpesvirus entry mediator (HVEM/TNFRSF14), a receptor expressed by T lymphocytes, is a recently identified member of the human and mouse TNF superfamily. TNFSF14/LIGHT/CD258 is a 29-kD type II transmembrane protein produced by activated T cells, as well as monocytes and granulocytes, and immature DCs. In vitro, HVEM/LIGHT immune checkpoint pathway induces potent CD28-independent costimulatory activity, leading to NF-kB activation, production of IFN-γ and other cytokines, and T cell proliferation in response to allogeneic DCs. In vivo blockade studies show HVEM/LIGHT immune checkpoint pathway is involved in promotion of cytolytic T cell responses to tumors and the development of GVHD, and transgenic overexpression of TNFSF14/LIGHT/CD258 within T cells leads to T cell expansion and causes various severe autoimmune diseases (Qunrui Ye et al. J Exp Med. 2002 Mar. 18; 195(6): 795-800).

CD28H is constitutively expressed on all naive T cells. B7 homologue 5 (B7-H5), was identified as a specific ligand for CD28H. B7-H5 is constitutively found in macrophages and could be induced on dendritic cells. The B7-H5/CD28H interaction selectively costimulates human T-cell growth and cytokine production via an AKT-dependent signalling cascade (Zhu Y et al., Nat Commun. 2013; 4:204).

OX40, also called CD134, has OX40L, or CD252, as its ligand. Like CD27, OX40 promotes the expansion of effector and memory T cells, however it is also noted for its ability to suppress the differentiation and activity of T-regulatory cells, and also for its regulation of cytokine production. OX40's value as a drug target primarily lies it the fact that, being transiently expressed after T-cell receptor engagement, it is only upregulated on the most recently antigen-activated T cells within inflammatory lesions. Anti-OX40 monoclonal antibodies have been shown to have clinical utility in advanced cancer (Weinberg A D, Morris N P, Kovacsovics-Bankowski M, Urba W J, Curti B D (Nov. 1, 2011). “Science gone translational: the OX40 agonist story”. Immunol Rev. 244 (1): 218-31).

GITR, short for Glucocorticoid-Induced TNFR family Related gene, prompts T cell expansion, including Treg expansion. The ligand for GITR (GITRL) is mainly expressed on antigen presenting cells. Antibodies to GITR have been shown to promote an anti-tumor response through loss of T_{reg} lineage stability (see Nocentini G, Ronchetti S, Cuzzocrea S, Riccardi C (May 1, 2007). “GITR/GITRL: more than an effector T cell co-stimulatory system”. Eur J Immunol. 37 (5): 1165-9).

ICOS, short for Inducible T-cell costimulator, and also called CD278, is expressed on activated T cells. Its ligand is ICOSL, expressed mainly on B cells and dendritic cells. The molecule seems to be important in T cell effector function (Burmeister Y, Lischke T, Dahler A C, Mages H W, Lam K P, Coyle A J, Kroczek R A, Hutloff A (Jan. 15, 2008). “ICOS controls the pool size of effector-memory and regulatory T cells”. J Immunol. 180 (2): 774-782).

Another stimulatory checkpoint molecules, which belongs to the B7-CD28 superfamily, are notably CD28 itself and TGMID2.

CD28 is constitutively expressed on almost all human CD4+ T cells and on around half of all CD8 T cells. Binding with its two ligands (CD80 and CD86, expressed on dendritic cells) prompts T cell expansion.

TMIGD2 (also called CD28 homolog), modulates T cell functions through interaction with its ligand HHLA2; a newly identified B7 family member. TMIGD2 protein is constitutively expressed on all naïve T cells and the majority of natural killer (NK) cells, but not on T regulatory cells or B cells (see Yanping Xiao and Gordon J. Freeman, “A new B7:CD28 family checkpoint target for cancer immunotherapy: HHLA2”, Clin Cancer Res. 2015 May 15; 21(10): 2201-2203).

CD137 ligand (CD137L; also known as 4-1BBL and TNFSF9) is mainly expressed on professional antigen-presenting cells (APCs) such as dendritic cells, monocytes/macrophages, and B cells, and its expression is upregulated during activation of these cells. However, its expression has been documented on a variety of hematopoietic cells and nonhematopoietic cells. Generally, 4-1BBL/CD137L is constitutively expressed on many types of cells but its expression levels are low except for a few types of cells. Interestingly, 4-1BBL/CD137L is coexpressed with CD137 (also known as 4-1BB and TNFRSF9) on various types of cells, but expression of CD137/4-1BB potently downregulates that of 4-1BBL/CD137L by cis-interactions between the two molecules resulting in endocytosis of 4-1BBL/CD137L (see Byungsuk Kwon et al. Is CD137 Ligand (CD137L) “Signaling a Fine Tuner of Immune Responses?” Immune Netw. 2015 June;15(3):121-124).

Finally other immune checkpoint molecules according to the invention also include CD244 (or 2B4) and SIRPα.

2B4/CD244 is a member of the signaling lymphocyte activation molecule (SLAM)-related receptor family and is also known as SLAMF4 and CD244. All members of the SLAM family share a similar structure, including an extracellular domain, a transmembrane region, and a tyrosine rich cytoplasmic region. 2B4 & CD48 Immune Checkpoint Pathway can lead to signaling through both receptors. CD48/SLAMF2 signaling in B cells leads to homotypic adhesion, proliferation and/or differentiation, release of inflammatory effector molecules and isotype class switching. In addition, all of these processes are also elicited in T cells via CD48/SLAMF2 ligation with the addition of promoting their activation and/or cytotoxicity. 2B4 signaling requires signaling lymphocyte activation molecule (SLAM)-associated protein (SAP) or EWS-activated transcript 2 (EAT-2; also called SH2D1B). In CD8 T cells and NK cells 2B4/CD244 has been reported to exert both positive and negative regulation (see also Sebastian Stark. “2B4 (CD244), NTB-A and CRACC (CS1) stimulate cytotoxicity but no proliferation in human NK cells”. Int. Immunol. 2006 18 (2): 241-247).

CD47 is a cell surface glycoprotein with a variety of functions including regulation of phagocytosis through binding to the macrophage and dendritic cell specific protein signal regulatory protein alpha (SIRP alpha). Binding of SIRP alpha to CD47, as SIRP alpha & CD47 immune checkpoint pathway, essentially sends a “don't eat me” message to macrophages by initiating signaling to inhibit phagocytosis. Increased expression of CD47 is proposed to be a mechanism through which cancer cells evade immune detection and phagocytosis. Targeting of CD47 on cancer cells with an anti-CD47 blocking antibody can promote phagocytosis by macrophages in vitro. Further, treatment with an anti-CD47 blocking antibody synergized with rituximab treatment to promote phagocytosis in vitro and to eliminate cancer cells in an in vivo xenograft model of non-Hodgkin lymphoma. Further results demonstrate that CD47 expression increases in a variety of human solid tumor types and that blocking the SIRP alpha & CD47 immune checkpoint pathway with an anti-CD47 antibody can promote phagocytosis of solid tumor cells in vitro and reduce growth of solid tumors in vivo (see Martina Seiffert et al. “Signal-regulatory protein a (SIRPα) but not SIRPβ is involved in T-cell activation, binds to CD47 with high affinity, and is expressed on immature CD34+CD38-hematopoietic cells”. 2001; Blood: 97 (9)).

As used herein, the expression “modulator of an immune checkpoint protein”, or “checkpoint regulator cancer immunotherapy agent” (both expressions can be used interchangeably in the sense of the invention) has its general meaning in the art and refers to any compound inhibiting the function of an immune inhibitory checkpoint protein (inhibitory immune checkpoint inhibitors, or immune checkpoint inhibitors as previously described) or stimulating the function of a stimulatory checkpoint protein (stimulatory immune checkpoint agonist or immune checkpoint agonist used interchangeably). Inhibition includes reduction of function and full blockade.

The immune checkpoint modulators include peptides, antibodies, fusion proteins, nucleic acid molecules and small molecules. For certain immune checkpoint protein (i.e., immune pathway gene products), the use of either antagonists or agonists of such gene products is also contemplated, as are small molecule modulators of such gene products.

Preferred immune checkpoint inhibitors or agonists are antibodies, or fusions proteins that specifically recognize immune checkpoint proteins or their ligands, as described previously.

A fusion protein for use as immune checkpoint modulator can be made by fusion of a checkpoint molecule as described above with the crystallizable fragment (Fc) region of an immunoglobulin. Preferably antibodies are monoclonal antibodies.

A number of immune checkpoint inhibitors and agonists are known in the art and in analogy of these known immune checkpoint protein modulators, alternative immune checkpoint modulators may be developed in the (near) future and be used in combination with an agonist of AHR according to the invention.

An immune checkpoint modulator according to the invention results in an activation of the immune system and in particular leads to an amplification of antigen-specific T cell response. In particular, the immune checkpoint modulator of the present invention is administered for enhancing the proliferation, migration, persistence and/or cytoxic activity of CD8+ T cells in the subject and in particular the tumor-infiltrating of CD8+ T cells of the subject. As used herein “CD8+ T cells” has its general meaning in the art and refers to a subset of T cells which express CD8 on their surface. They are MHC class I-restricted, and function as cytotoxic T cells. “CD8+ T cells” are also called CD8+ T cells are called cytotoxic T lymphocytes (CTL), T-killer cell, cytolytic T cells, CD8+ T cells or killer T cells. CD8 antigens are members of the immunoglobulin supergene family and are associative recognition elements in major histocompatibility complex class I-restricted interactions. The ability of the immune checkpoint modulator to enhance T CD8 cell killing activity may be determined by any assay well known in the art. Typically said assay is an in vitro assay wherein CD8+ T cells are brought into contact with target cells (e.g. target cells that are recognized and/or lysed by CD8+ T cells).

For example, the immune checkpoint modulator of the present invention can be selected for the ability to increase specific lysis by CD8+ T cells by more than about 20%, preferably with at least about 30%, at least about 40%, at least about 50%, or more of the specific lysis obtained at the same effector: target cell ratio with CD8+ T cells or CD8 T cell lines that are contacted by the immune checkpoint inhibitor of the present invention, Examples of protocols for classical cytotoxicity assays are conventional.

The at least one immune checkpoint modulator according to the invention can be a modulator of an inhibitory immune checkpoint molecule and/or of a stimulatory immune checkpoint molecule.

For example, the checkpoint regulator cancer immunotherapy agent can be an agent which blocks (an antagonist of) an immunosuppressive receptor (i.e., an inhibitory immune checkpoint) expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

In some embodiments, the checkpoint blockade cancer immunotherapy agent is selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, anti-B7H6 antibodies, anti-CD86 antibodies, anti-Gal9 antibodies, anti-HVEM antibodies, anti-CD28 antibodies, anti-A2aR antibodies, anti-CD80 antibodies, anti-KIR(s) antibodies, A2aR drugs (notably adenosine analogs), anti-DCIR (C-type lectin surface receptor) antibodies, anti-ILT3 antibodies, anti-ILT4 antibodies, anti-CD31 (PECAM-1) antibodies, anti-CD39 antibodies, anti-CD73 antibodies, anti-CD94/NKG2 antibodies, anti-GP49b antibodies, anti-KLRG1 antibodies, anti-LAIR-1 antibodies, anti-CD305 antibodies, and their combinations. In certain embodiments, the checkpoint blockade cancer immunotherapy agent is an anti-PD-1 or an anti-PD-L1 antibody.

Examples of anti-CTLA-4 antibodies are described in U.S. Pat. Nos: 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4.

Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211).

Other immune-checkpoint inhibitors include B7 inhibitors, such as B7-H3 and B7-H4 inhibitors, notably, the anti-B7-H3 antibody MGA271 (Loo et al., 2012, Clin. Cancer Res. July 15 (18) 3834).

Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9, its natural ligand. Antibodies having specificity for TIM-3 are well known in the art and typically those described in WO2011155607, WO2013006490 and WO2010117057.

In some embodiments, the immune checkpoint inhibitor is an Indoleamine 2,3-dioxygenase (IDO) inhibitor, preferably an IDO1 inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′- diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is an anti-TIGIT (T cell immunoglobin and ITIM domain) antibody.

In some embodiments, the immune checkpoint inhibitor is an anti-VISTA antibody, preferably a monoclonal antibody (Lines J L, Sempere L F, Wang L, et al. VISTA is an immune checkpoint molecule for human T cells. Cancer research. 2014; 74(7):1924-1932. doi:10.1158/0008-5472.CAN-13-1504).

In a preferred embodiment, the checkpoint modulator cancer immunotherapy agent is a CTLA4 blocking antibody, such as Ipilimumab, a PD-1 blocking antibody, such as Nivolumab or Pembrolizumab, a PDL-1 blocking antibody or a combination thereof. Typically, the checkpoint modulator cancer immunotherapy agent is a PD-1 blocking antibody, such as Nivolumab or Pembrolizumab, or a PDL-1 blocking antibody.

The checkpoint modulator cancer immunotherapy agent can also be an agent, which activates a stimulatory immune checkpoint receptor expressed by activated T lymphocytes, or by NK cells, or an agent which mimics the principal ligands of these receptors, and results also in the amplification of antigen-specific T cell responses.

Thus, the checkpoint modulator cancer immunotherapy agent can typically be an agonistic antibody, notably a monoclonal agonistic antibody to a stimulatory immune checkpoint molecules as described above, for example selected from the group consisting of agonistic anti -4-1BB, -OX40, -GITR, -CD27, -ICOS, -CD40L, -TMIGD2, -CD226, -TNFSF25, -2B4 (CD244), -CD48, -B7-H6 Brandt (NK ligand), -CD28H -LIGHT (CD258, TNFSF14), and -CD28 antibodies.

The checkpoint agonist cancer immunotherapy agent can also be a fusion protein for example, a 4-1BB-Fc fusion protein, an Ox40-Fc fusion protein, a GITR-Fc fusion protein, a CD27-Fc fusion protein, an ICOS-Fc fusion protein, a CD40L-Fc fusion protein, a TMIGD2-Fc fusion protein, a CD226-Fc fusion protein, a TNFSF25-Fc fusion protein, a CD28-Fc fusion protein, a 2B4 (CD244) fusion protein, a CD48 fusion protein, a B7-H6 Brandt (NK ligand) fusion protein, a CD28H fusion protein and a LIGHT (CD258, TNFSF14) fusion protein.

Several of the 4-1BB agonists show great potential for application to human cancers. For example, BMS-666513, a fully humanized mAb against 4-1BB, has completed phase I and II trials for its anticancer properties in patients with melanoma, renal cell carcinoma, and ovarian cancer (Sznol M, Hodi F S, Margolin K, McDermott D F, Ernstoff M S, Kirkwood J M, et al. Phase I study of BMS-663513, a fully human anti-CD137 agonist monoclonal antibody, in patients (pts) with advanced cancer (CA). J Clin Oncol 26: 2008 (May 20 suppl; abstr 3007). Seven OX40 agonists are now in development, 6 of which take the form of fully human monoclonal antibodies to address the mouse antibody issue. One OX40L-Fc fusion protein, MED16383, is also undergoing clinical evaluation; this links 2 OX40L molecules to part of the fragment crystallizable (Fc) region of immunoglobulin. In preclinical testing, the fusion protein appears to have stronger effects than OX40 antibodies, possibly because it may also activate dendritic cells and vascular endothelial cells in addition to T cells. Examples of Ox40 agonists include MEDI6469, MEDI6383, MEDI0652, PF-04515600, MOXP0916, GSK3174998, INCAGNO 1949.

Agonistic antibodies to GITR have been developed such as a humanized anti-human GITR mAb (TRX518. Tolerx Inc. Agonistic antibodies to human glucocorticoid-induced tumor necrosis factor receptor as potential stimulators of T cell immunity for the treatment of cancer and viral infections. Expert Opin Ther Patents. 2007;17:567-575, see also Schaer D A, Murphy J T, Wolchok J D. Modulation of GITR for cancer immunotherapy. Curr Opin Immunol. 2012 April;24(2):217-24).

An example of an agonistic antibody to CD27, another member of the TNF family include the fully human 1F5 mAb that is now in Phase I clinical testing in B-cell malignancies, melanoma and renal cell carcinoma as CDX-1127 (varlilumab) (Analysis of the properties of the anti-CD27 monoclonal antibody (mAb) that is currently in clinical trials (Vitale L A, He L-Z, Thomas L J et al. 2012 Development of a human monoclonal antibody for potential therapy of CD27-expressing lymphoma and leukemia. Clin. Cancer Res. 18(14), 3812-3821).

Initial clinical trials of agonistic CD40 mAb have shown highly promising results in the absence of disabling toxicity, in single-agent studies. To date, four CD40 mAb have been investigated in clinical trials: CP-870,893 (Pfizer and VLST), dacetuzumab (Seattle Genetics), Chi Lob 7/4 (University of Southampton), and lucatumumab (Novartis) (Vonderheide R H, Flaherty K T, Khalil M, Stumacher M S, Bajor D L, Hutnick N A, et al. Clinical activity and immune modulation in cancer patients treated with CP-870,893, a novel CD40 agonist monoclonal antibody. J Clin Oncol. 2007;25:876-83; Khubchandani S, Czuczman M S, Hernandez-Ilizaliturri F J. Dacetuzumab, a humanized mAb against CD40 for the treatment of hematological malignancies. Curr Opin Investig Drugs. 2009;10:579-87; Johnson P W, Steven N M, Chowdhury F, Dobbyn J, Hall E, Ashton-Key M, et al. A Cancer Research UK phase I study evaluating safety, tolerability, and biological effects of chimeric anti-CD40 monoclonal antibody (MAb), Chi Lob 7/4. J Clin Oncol. 2010;28:2507; Bensinger W, Maziarz R T, Jagannath S, Spencer A, Durrant S, Becker P S, et al. A phase 1 study of lucatumumab, a fully human anti-CD40 antagonist monoclonal antibody administered intravenously to patients with relapsed or refractory multiple myeloma. Br J Haematol. 2012;159:58-66).

The checkpoint agonist cancer immunotherapy agent can also be an anti-ICOS agonist monoclonal antibody (Kutlu Elpek, Christopher Harvey, Ellen Duong, Tyler Simpson, Jenny Shu, Lindsey Shallberg, Matt Wallace, Sriram Sathy, Robert Mabry, Jennifer Michaelson, and Michael Briskin, Abstract A059: Efficacy of anti-ICOS agonist monoclonal antibodies in preclinical tumor models provides a rationale for clinical development as cancer immunotherapeutics; Abstracts: CRI-CIMT-EATI-AACR Inaugural International Cancer Immunotherapy Conference: Translating Science into Survival; Sep. 16-19, 2015; New York, N.Y.), or an anti-CD28 agonist antibody (for use notably in combination with anti-PD-1 immunotherapy, see T cell costimulatory receptor CD28 is a primary target for PD-1-mediated inhibition) see also Melero I, Hervas-Stubbs S, Glennie M, Pardoll D M, Chen L. Nat Rev Cancer. 2007 February;7(2):95-106, for review.

According to the present invention more than one modulator of an immune checkpoint protein can be used in combination with an AHR agonist according to the present invention. For example, at least one modulator of an inhibitory immune checkpoint inhibitor (notably selected from an anti-PD-1, an anti-PD-L1, an anti-CTLA-4 and their combinations) can be used in combination with at least one stimulatory immune checkpoint agonist as mentioned above. Co-stimulatory and co-inhibitory immune checkpoint molecules are notably described in the review of Chen L & Flies B (Nat rev Immuno., 2013 mentioned above).

Preferably according to the invention an AHR agonist, notably a tryptophan metabolite or dietary AHR agonist, most preferably an dietary indole AHR agonist is used in combination with at least one immune checkpoint modulator, notably selected from an anti-PD-1, an anti-PD-L1, an anti-CTLA-4 and their combinations.

### Patients

Typically, the patient according to the invention is a mammalian, preferably a human.

Typically said patient is suffering from a cancer, or is in remission or is at risk of a cancer.

The cancer may be a solid cancer or a cancer affecting the blood (i.e., leukemia). Leukemia include for example acute myelogenous leukemia (AML), chronic myelogenous leukemia (CML), acute lymphocytic leukemia (ALL), and chronic lymphocytic leukemia, (including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland, sarcomas such as liposarcoma and soft-tissue sarcoma. Skin cancers comprise melanoma, nonmelanoma skin cancer.

### Dosage

Preferably the AHR agonist and the immune checkpoint modulator are in an effective dose. Typically the combined treatment regimen of the invention (i.e., AHR agonist and the at least one immune checkpoint modulator) is therapeutically effective.

Currently available therapies and their dosages, routes of administration and recommended usage are known in the art and have been described in such literature as the Physician's Desk Reference (60th ed., 2006). Routes of administration include parenterally, intravenously, subcutaneously, intracranially, intrahepatically, intranodally, intraureterally, subureterally, subcutaneously, and intraperitoneally.

Preferably the AHR agonist as per the invention is in the form of a composition suitable for oral or enteral, preferably oral, administration.

In the various embodiments of the present invention, the pharmaceutical composition comprising the AHR agonist and/or the immune checkpoint modulator further comprises a pharmaceutically acceptable carrier and/or vehicle. Typically the AHR agonist and the immune checkpoint modulator are in separate compositions.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with enveloped viruses and do not prevent virus entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS).

Dosage of one or more agents of the invention (e.g., the AHR agonist and immune checkpoint modulator) can be determined by one of skill in the art and can also be adjusted by the individual physician in the event of any complication.

The effective dose is determined and adjusted depending on factors such as the composition used, the route of administration, the physical characteristics of the individual under consideration such as sex, age and weight, concurrent medication, and other factors, that those skilled in the medical arts will recognize. Dosage of the AHR agonist may be empirically determined such as a significant improvement in the patient condition is observed when associated with the immune checkpoint therapy.

### Combination Therapies

In a specific embodiment, cycling therapy involves the administration of a first cancer therapeutic for a period of time, followed by the administration of a second cancer therapeutic for a period of time, optionally, followed by the administration of a third cancer therapeutic for a period of time and so forth, and repeating this sequential administration, i.e., the cycle in order to reduce the development of resistance to one of the cancer therapeutics, to avoid or reduce the side effects of one of the cancer therapeutics, and/or to improve the efficacy of the cancer therapeutics.

When two the two combined treatment according to the invention are administered to a patient concurrently, typically in a therapeutically effective regimen the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the two therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic as per the invention, to a patient in need thereof.

For example, the AHR agonist (notably in the form of a medical food or a dietary supplement) is administered chronically or over long time schedules and the immune checkpoint modulator is administered in a sequential schedule.

Preferably the combined administration of the AHR agonist with an immune checkpoint modulator according to the invention leads to a synergistic anti-cancer effect.

### Kit of Parts Preparations

The present application also encompasses preparations containing an AHR agonist as previously described and at least one immune checkpoint modulator as also described above, as a combined preparation for simultaneous, separate or sequential use in cancer treatment. According to such preparations in the form of kit-of-parts” the individual active compounds (i.e., the AHR agonist and the at least one immune checkpoint modulator), represent therapeutic agents and are physically separated, provided that the use of those compounds, either simultaneously, separately or sequentially, produces the new and unexpected joint therapeutic effect as herein described that is not attained by the compounds independently of each other. Indeed as demonstrated by the results below, the claimed combination of active ingredients did not represent a mere aggregate of known agents, but rather a new combination with the surprising, valuable property that the combined anti-tumor effect is much more important that the simple addition of the anti-tumor effects that are observed, when those active ingredients are used separately.

Both active ingredients may be thus formulated into separate compositions or into a unique composition. Preferably the one or more AHR agonist and the one or more immune checkpoint modulator are formulated in separate compositions.

The therapeutic agents as per the invention can be suitably formulated and introduced into a subject or the environment of the cell by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral, enteral, transdermal (topical), transmucosal, and rectal administration.

### Method of Treatment

The present invention also relates to a method for treating a patient suffering from cancer, wherein said method comprises the combined administration of an effective amount of one or more AHR agonist and an effective amount of at least one immune checkpoint modulator as described previously.

Typically, said combined administration is administered according to a therapeutically effective regimen.

The invention will further be illustrated in view of the following examples.

### BRIEF DESCRIPTION OF THE FIGURES

FIG. 1: IRF4 and MafB are essential for mo-DC and mo-Mac differentiation. (A) Volcano plot showing the fold change and significance of transcription factor genes between ascites mo-DC and mo-Mac. Genes not expressed in in vitro-generated mo-DC or mo-Mac were filtered out. Adjusted p values determined by differential expression analysis. (B) Cell-sorted ascites mo-DC and mo-Mac, and blood CD14⁺ monocytes (Mono) were analyzed by Immuno Blot. Representative of 4 independent experiments. (C) CD14⁺ monocytes were cultured with MCSF, IL-4 and TNF-α for 3 h, 6 h, 12 h or 24 h, or processed directly after isolation (0). IRF4 and MAFB expression were analyzed by RT-qPCR in total cells. Each color represents an individual donor (n=5 in 3 independent experiments). (D) CD14⁺ monocytes were cultured with MCSF, IL-4 and TNF-α for 5 days. Total cells were lyzed at different days and analyzed by Immuno Blot. Representative of 5 independent experiments. (E-H) Monocytes were infected at day 0 with lentivirus containing sh RNA against IRF4 (E-F), or MAFB (G-H), or control sh RNA. After 5 days of culture, cells were analyzed by Immuno Blot (E and G), or by flow cytometry (F and H). (E and G) Silencing quantified based on Immuno Blot stainings. (F and H) Proportions of DC and macrophages in the culture at day 5. Each symbol represents an individual donor (F n=6 in 3 independent experiments, H n=8 in 4 independent experiments). *p<0.05, **p<0.01.

FIG. 2: AHR is a molecular switch for mo-DC versus mo-Mac differentiation. (A-B) CD14⁺ monocytes were infected at day 0 with lentivirus containing sh RNA against AHR, or control sh RNA. After 5 days of culture, cells were analyzed by Immuno Blot (A) or by flow cytometry (B). (A) Silencing quantified based on Immuno Blot stainings. (B) Proportions of DC and macrophages in the culture at day 5. Each symbol represents an individual donor (n=6 in 3 independent experiments). *p<0.05. (C) Monocytes were cultured with MCSF, IL-4 and TNF-α for 5 days, in the presence of various concentrations of FICZ (AHR agonist) or SR1 (AHR inhibitor). Proportions of DC and macrophages at day 5. Each symbol represents an individual donor (n=10 in 5 independent experiments for FICZ and n=8 for SR1 in 4 independent experiments). (D-E) CD14⁺ monocytes were analyzed directly after isolation, or were cultured for 3h in medium alone or with various combinations of MCSF, IL-4, TNF-α, FICZ or SRI. (D) Relative expression of IRF4 and MAFB measured by RT-qPCR. Box plots representing the 5-95 percentile (n=6 in 3 independent experiments). (E) Expression of IRF4 and MAFB at the single cell level was measured by fluorescent in situ hybridization coupled to flow cytometry. Proportions of IRF4⁺ and MAFB⁺ cells. Each symbol represents an individual donor (n=6 in 3 independent experiments). *p<0.05, **p<0.01, ***p<0.001.

FIG. 3: AHR acts on mo-DC differentiation through BLIMP-1. (A) CD14⁺ monocytes were cultured with MCSF, IL-4 and TNF-α for 3 h, 6 h, 12 h or 24 h in the presence or absence of FICZ or SR1, or processed directly after isolation (0). PRDM1 expression measured by RT-qPCR. Each color represents an individual donor (n=5 in 3 independent experiments). (B) Monocytes were cultured with MCSF, IL-4 and TNF-α for 5 days. Total cells were lyzed at different days and analyzed by Immuno Blot. Representative of 4 independent experiments. (C-E) Monocytes were infected at day 0 with lentivirus containing sh RNA against PRDM1, or control sh RNA. After 5 days of culture, cells were analyzed by Immuno Blot (C) or by flow cytometry (D-E). (C) Silencing quantified based on Immuno Blot stainings. (D) Proportions of DC and macrophages in the culture at day 5. Each symbol represents an individual donor (n=8 in 4 independent experiments). *p<0.05. **p<0.01. (E) Cells were cultured in presence or absence of 62 nM FICZ. Proportions of DC and macrophages in the culture at day 5. Each symbol represents an individual donor (n=6 in 3 independent experiments). *p<0.05.

FIG. 4: AhR is involved in mo-DC differentiation in vivo in the mouse. (A-C) Ear skin from individual AhR^{-/-} mice or WT littermates (A-B), or C57BL/6 mice fed with an experimental diet supplemented or not with indole-3-carbinol (I3C) (C), was digested to prepare single-cell suspensions. After gating on live CD45⁺CD3⁻NK1.1⁻CD19⁻Ly6G⁻CD24⁻ CD11b⁺ cells, cells were separated into 5 subsets based on the expression of Ly6C, CD64, MHC II and CCR2. (A) One representative AhR^{-/-} and WT mouse is shown. (B-C) Proportions of monocytes, early mo-DC, mo-DC, MHC II⁺ macrophages and resident MHC II⁻ macrophages among Ly6C⁺ or CD64⁺ cells. Each symbol represents an individual mouse (B n=9 in 2 independent experiments, C n=12 in 2 independent experiments).*p<0.05, **p<0.01. (D-E) Cells from the peritoneal lavage were analyzed by flow cytometry. After gating on live CD115⁺CD11b⁺ cells, cells were separated into MHCII⁺CD226⁺ cells and ICAM2⁺MHCII⁻ cells. (D) Cell-sorted cells were analyzed after cytospin and Giemsa/May-Grünwald staining. Bar=10 μm. Representative of 2 independent experiments. (E) C57BL/6 mice fed with an experimental diet supplemented or not with I3C, and treated or not with a cocktail of antibiotics. Percentage of MHCII⁺CD226⁺ mo-DC among CD115⁺CD11b⁺ cells is shown. Each symbol represents an individual mouse (n=6 in 2 independent experiments). *p<0.05, **p<0.01, ****p<0.0001.

FIG. 5: AhR agonist improves the efficacy of anti-PD1 treatment in tumor-bearing mice. Mice were injected subcutaneously with B16-OVA (A-B) or MCA101-OVA (C-D) tumor cells. Tumor growth was monitored twice a week. Mice were fed with a control diet or supplemented with indole-3-carbinol (I3C). Mice were treated with PBS (vehicle control) or with anti-PD1 antibody. (A-B) B16-OVA tumor growth. Mean+/−SEM (n=12 for A, n=8 for B). (C) MCA-OVA tumor growth. Mean+/−SEM (n=16). (D) Number of mice showing complete tumor rejection, tumor regression or tumor progression, for each treatment.

### EXAMPLES

### Example 1: Aryl Hydrocarbon Receptor Controls Monocyte Differentiation into Dendritic Cells Versus Macrophage

### Introduction

Mononuclear phagocytes are divided into three groups: macrophages, monocytes and dendritic cells (DC). Macrophages derive from embryonic precursors whose differentiation is strongly imprinted by the micro-environment (Gosselin et al., 2014; Haldar et al., 2014; Lavin et al., 2014; Okabe and Medzhitov, 2014). By contrast, classical DC derive from pre-committed precursors that follow a pre-determined developmental program primed at an early stage, independently of their tissue of residence (Breton et al., 2016; Schlitzer et al., 2015). When entering tissues, monocytes can differentiate into either macrophages or DC (Mildner et al., 2013; Segura and Amigorena, 2013). Whether mo-DC and mo-Mac represent variations of one highly plastic cell type or distinct bona fide lineages remains unclear (Guilliams et al., 2014). In addition, what environmental cues drive monocyte fate towards mo-Mac versus mo-DC and what molecular regulators orchestrate this process remains to be established.

In mouse models, mo-DC and mo-Mac appear during inflammation but are also found in the steady-state at mucosal sites such as intestine and skin (Bain et al., 2014; Mildner et al., 2013; Segura and Amigorena, 2013; Tamoutounour et al., 2013). There is strong experimental evidence that the same scheme applies to humans. Monocyte-derived cells are found in the steady-state in human skin (McGovern et al., 2014). In inflammatory conditions, monocyte recruitment is observed in the gut of inflammatory bowel disease patients (Grimm et al., 1995), in cantharidin-induced skin blisters (Jenner et al., 2014) and in the nasal mucosa of subjects with induced allergic rhinitis (Eguiluz-Gracia et al., 2016). Inflammatory macrophages and DC have been described in atopic dermatitis (Wollenberg et al., 1996), Crohn's disease (Bain et al., 2013; Kamada et al., 2008), psoriasis (Zaba et al., 2009), allergic rhinitis (Eguiluz-Gracia et al., 2016), rheumatoid arthritis and tumor ascites (Segura et al., 2013). Transcriptomic analysis shows that ascites DC share gene signatures with in vitro-generated monocyte-derived cells, supporting the idea that these DC represent tissue mo-DC (Segura et al., 2013).

We hypothesized that transcription factors differentially expressed between human mo-DC and mo-Mac may be involved in their differentiation from monocytes. In this study, we have identified candidate transcription factors by comparative transcriptomic analysis, and established a model of human monocyte differentation to test the role of these candidates. We found that IRF4 and MAFB were required for mo-DC and mo-Mac differentiation, respectively. We also have shown that AHR is essential for driving monocyte differentiation towards mo-DC. Finally, we validated the role of AHR in mo-DC differentiation in vivo in a mouse model and by analyzing clinical data from leprosy patients.

### Results

### Culturing Human Monocytes with M-CSF, IL-4 and TNF-α Yields mo-DC and mo-Mac Resembling Those Found in Tumor Ascites

To address the ontogeny of human monocyte-derived cells, we first searched for transcription factors that are differentially expressed between monocytes, ascites mo-DC and ascites mo-Mac. Using our transcriptomic data (Segura et al., 2013), we established a list of candidates. To test their role, we turned to an in vitro model of monocyte differentiation. Current culture models yield either mo-DC or mo-Mac depending on cytokines used. By contrast, we needed a model that would mimic the differentiation of monocytes into mo-DC and mo-Mac in the same environment. We therefore established a new in vitro system enabling the differentiation of both mo-DC and mo-Mac in the same culture.

Macrophage colony-stimulating factor (M-CSF) and its receptor are essential for mo-DC and mo-Mac differentiation in vivo during inflammation in mice (Davies et al., 2013; Greter et al., 2012), and M-CSFR is highly expressed on human mo-DC and mo-Mac found in vivo (McGovern et al., 2014; Segura et al., 2013). Therefore, we designed a cytokine cocktail based on M-CSF or IL-34, the two ligands of M-CSFR. We also included IL-4, a cytokine known to induce the expression by cultured monocytes of CD1 molecules, which are highly expressed on ascites mo-DC (Segura et al., 2013). Finally, we added TNF-α, a major mediator of inflammation. Culturing human blood CD14⁺ monocytes with M-CSF, IL-4 and TNF-αor IL-34, IL-4 and TNF-α yielded in the same culture two main populations expressing CD16 or CD1a, and displaying a typical macrophage or DC morphology, respectively. Only CD1a⁺ cells could efficiently induce allogeneic naive CD4⁺ T cell proliferation, confirming that they were bona fide DC. We then characterized the properties of cultured mo-DC and mo-Mac compared to that of ascites mo-DC and mo-Mac. Both mo-DC and mo-Mac secreted IL-6 after stimulation with CD40L, but only mo-DC secreted IL-23, similar to what we observed with ascites cells (Segura et al., 2013). Phenotypic comparison of mo-DC and mo-Mac differentiated with M-CSF, or IL-34, IL-4 and TNF-α with ascites mo-DC and mo-Mac showed similar expression for various surface markers, except for CD14 which was down-regulated upon culture. CD14⁺ monocytes were routinely isolated by positive selection using magnetic beads with 90-95% purity, contaminating cells being CD14⁺CD16⁺ monocytes. To address whether the presence of CD14⁺CD16⁺ monocytes could influence the culture outcome, we isolated highly pure CD14⁺ monocytes by cell sorting. The absence of contaminating CD14⁺CD16⁺ monocytes did not impact monocyte differentiation into both mo-DC and mo-Mac. In addition, CD16⁺ monocytes (isolated using magnetic beads) had a low survival rate and did not maintain CD16 expression in culture, suggesting that potential contaminating CD16⁺ monocytes had a negligeable effect on the final proportions of mo-DC and mo-Mac. Of note, monocytes differentiated with GM-CSF and IL-4, a widely used culture system, yielded only CD1a⁺ mo-DC. The phenotype of mo-DC derived with GM-CSF and IL-4, with or without TNF-α, was less similar to that of ascites mo-DC. When stimulated with a toll-like receptor (TLR)7 and TLR8 ligand (R848) and an endogenous danger signal (uric acid cristals), mo-DC differentiated with M-CSF, or IL-34, IL-4 and TNF-α secreted high concentrations of inflammatory cytokines (IL-1α and IL-6), consistent with the secretory ability of ascites mo-DC (Segura et al., 2013). Of note, mo-DC differentiated with GM-CSF and IL-4 were less efficient for the secretion of IL-1β and IL-6, although they secreted TNF-α and the chemokine CXCL10 at similar concentrations as mo-DC differentiated with M-CSF, or IL-34, IL-4 and TNF-α.

To determine whether these populations had a stable phenotype over time, we sorted CD1a⁻CD16⁻ cells, mo-DC and mo-Mac after 5 days of culture and re-cultured them separately with M-CSF, IL-4 and TNF-α. After 2 days of re-culture, the phenotype of CD1a⁻ CD16⁻ cells, mo-DC or mo-Mac remained stable based on the expression of CD16, CD163 and CD1a. After 4 days of re-culture, only CD1a⁻CD16⁻ cells and mo-Mac were still viable, and their phenotype was largely unchanged. These results show that mo-DC and mo-Mac are maintained as stable populations over the course of the culture, and that mo-DC or mo-Mac do not emerge from CD1a⁻CD16⁻ cells at later time points. To address whether a single monocyte could give rise to both mo-DC and mo-Mac in our culture model, we stained monocytes with a proliferation dye and analyzed cell proliferation after 5 days. As a positive control, we stimulated monocytes with the mitogen phytohaemagglutin-L (PHA-L), which induced the proliferation of a portion of monocytes. Monocytes did not proliferate in the culture, suggesting that there was a precursor-product relationship between a single monocyte and a single mo-DC, or mo-Mac, progeny.

Finally, to complete the characterization of our culture model, we compared the transcriptome of cell-sorted mo-DC and mo-Mac differentiated with M-CSF, or IL-34, IL-4 and TNF-α with that of cell-sorted ascites mo-DC and mo-Mac, blood CD14⁺ monocytes, blood CD1c⁺ DC and mo-DC differentiated with GM-CSF and IL-4. Supervised analysis of the micro-array data showed differential expression for selected phenotypic markers as expected. Comparative transcriptomic analysis showed that mo-Mac and mo-DC differentiated with M-CSF, IL-4 and TNF-α were highly similar to those differentiated with IL-34, IL-4 and TNF-α. In addition, these in vitro-generated mo-Mac and mo-DC clustered close to ascites mo-Mac and mo-DC respectively, while the transcriptome of mo-DC differentiated with GM-CSF and IL-4 was closer to that of blood CD1c⁺ DC. These results show that our culture system yields mo-DC and mo-Mac populations that closely resemble mo-DC and mo-Mac present in human tumor ascites. For the rest of the study, we used monocytes cultured with M-CSF, IL-4 and TNF-α as a model to analyze monocyte-derived cell differentiation.

### IRF4 and MAFB are Essential for the Development of mo-DC and mo-Mac

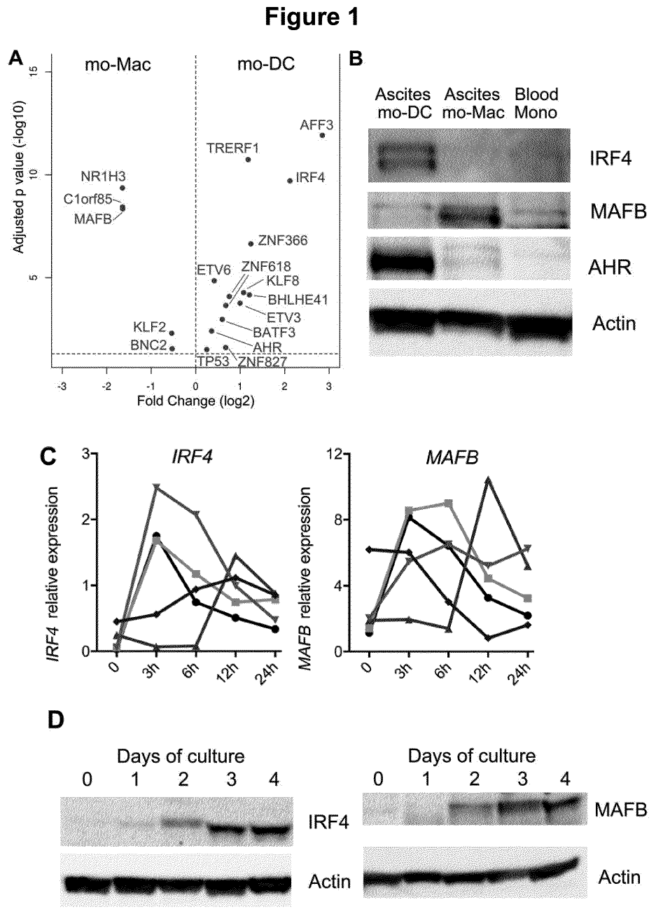

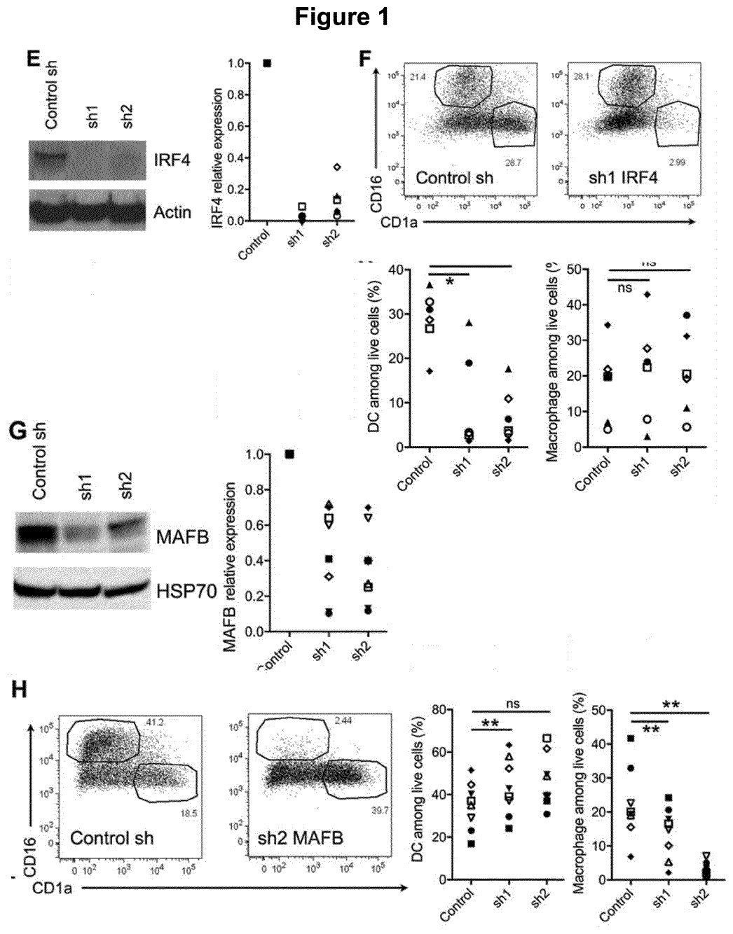

To refine our list of candidates for the differentiation of monocyte-derived cells, we removed transcription factors that were not expressed or not differentially expressed in in vitro-derived mo-DC versus in vitro-derived mo-Mac (FIG. 1A). We selected for further validation two candidates, IRF4 and MAFB, previously proposed to be involved in the development of a subset of mouse classical DC (Murphy et al., 2015), and in macrophage differentiation (Kelly et al., 2000) respectively. IRF4 and MAFB were differentially expressed in mo-DC versus mo-Mac, both in vitro and in vivo. In addition, we confirmed the differential expression of IRF4 and MAFB in blood monocytes, ascites mo-DC and ascites mo-Mac at the protein level (FIG. 1B).

IRF4 and MAFB were expressed early during the culture both at the mRNA (FIG. 1C) and protein levels (FIG. 1D), consistent with their possible role as master regulator transcription factors. To address the role of IRF4, we silenced its expression by infecting monocytes at the start of the culture with lentiviral vectors containing shRNA against IRF4, or control shRNA (FIG. 1E). Inhibition of IRF4 expression induced a dramatic reduction of mo-DC while maintaining the mo-Mac population (FIG. 1F). We used a similar strategy to analyze the role of MAFB (FIG. 1G). Silencing of MAFB resulted in a strong decrease in mo-Mac and an increase in mo-DC differentiation (FIG. 1H). Analysis of additional phenotypic markers confirmed the disappearance of mo-DC or mo-Mac from the culture, rather than the mere down-regulation of CD1a or CD16 expression. These results show that IRF4 and MAFB are essential for mo-DC and mo-Mac development, respectively.

### Monocytes are not Heterogeneous for the Expression of mo-DC Gene Signatures

These results could be explained either by the presence of two distinct precursor populations or by the existence among blood CD14⁺ monocytes of two transcriptionally primed populations that would be pre-committed to become mo-DC or mo-Mac (Schlitzer et al., 2015). To address the heterogeneity of CD14⁺ monocytes, we performed single-cell RNA-seq on CD14⁺ monocytes from 2 donors isolated using magnetic beads. Cell purity as assessed by flow cytometry was 93% and 95% respectively. We generated single-cell transcriptomes using a droplet-based method enabling 3′ mRNA counting (Zheng et al., 2017). To evaluate the heterogeneity of CD14⁺ monocytes, we clustered cells using a graph-based approach with the Seurat package, which combines dimensionality reduction and graph-based partitioning algorithms for unsupervised clustering (Satija et al., 2015). For vizualisation of the cell clusters, we used t-Distributed Stochastic Neighbor Embedding (t-SNE). We found two clusters, one of which represents around 25% of total cells and corresponds to cells expressing FCGR3A (encoding CD16), high amounts of MHC class II molecules and several genes preferentially expressed in CD14⁺CD16⁺ and CD16⁺ monocytes including IFITM2 and IFITM3 (Villani et al., 2017; Wong et al., 2011). In addition to contaminating CD14⁺ CD16⁺ monocytes, some of these cells may correspond to CD14⁺ monocytes en route to differentiating into CD14⁺CD16⁺ monocytes (Patel et al., 2017). To address the potential heterogeneity of the CD16⁻ cluster, we performed a second analysis excluding cells from the CD16⁺ cluster. We did not detect subgroups of transcriptionally distinct cells within the CD16⁻ cluster, confirming that the CD14⁺ monocyte population is likely homogeneous, as previously reported (Villani et al., 2017). To confirm these results, we sought to address whether subpopulations of monocytes displayed transcriptional similarity with mo-DC or mo-Mac. Because of the limited number of genes detected per cell in our analysis (average of 1185), we interrogated published single-cell RNA-seq data obtained using the Smart-seq2 approach (Villani et al., 2017), characterized by a lower number of cells analyzed but a higher number of genes per cell (average of 5326). We defined gene signatures for mo-DC and mo-Mac using our transcriptomic data by identifying genes that were (i) more expressed in ascites mo-DC than in blood monocytes, (ii) more expressed in ascites mo-DC than in ascites mo-Mac, and (iii) more expressed in in vitro-derived mo-DC than in in vitro-derived mo-Mac (and vice-versa for mo-Mac). We then queried genes with at least a 2-fold change. Among these, 35 genes for mo-DC and 35 genes for mo-Mac were expressed in the single-cell RNA-seq data set. While none of the monocytes expressed the mo-DC signature, the mo-Mac signature was partially expressed by all monocyte subsets. We also assessed the expression of selected genes that could be involved in determining monocyte fate, including receptors for the cytokines used in our model (CSF1R, IL4R, TNFRSF1A), candidate transcription factors (IRF4, MAFB, AHR) or genes recently proposed to distinguish “DC-biased” monocytes in the mouse (FLT3, SPI1) (Menezes et al., 2016). While IRF4 was not expressed by monocytes, MAFB was detected in all monocyte subsets. We conclude that human CD14⁺ monocytes are not heterogeneous in their expression of mo-DC transcriptional signature. While monocytes do not contain a subpopulation that would be pre-committed towards mo-DC differentiation, they all express a partial mo-Mac gene signature, including MAFB, suggesting a default differentiation pathway towards mo-Mac if no other environmental triggers are encountered.

### AHR is a Molecular Switch for Monocyte Fate

Given that monocytes do not seem to be transcriptionally primed, we then hypothesized that environmental signals play a major role in driving monocyte fate. Among candidate transcription factors (FIG. 1A), we identified AHR, a ligand-activated transcription factor sensing tryptophan catabolites and metabolites generated by dietary intake, UV exposure, or microbiota (Stockinger et al., 2014). AHR was differentially expressed by mo-DC and mo-Mac at the mRNA and protein levels (FIG. 1B).

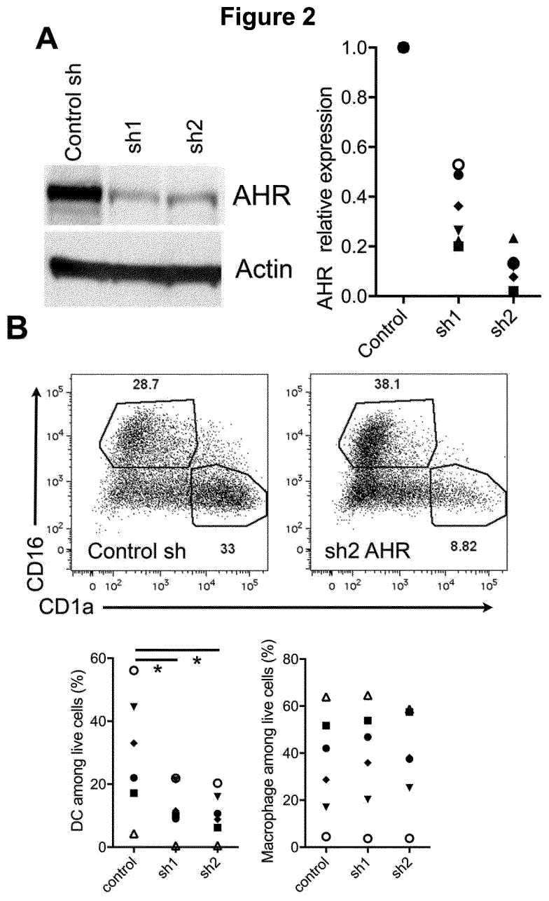

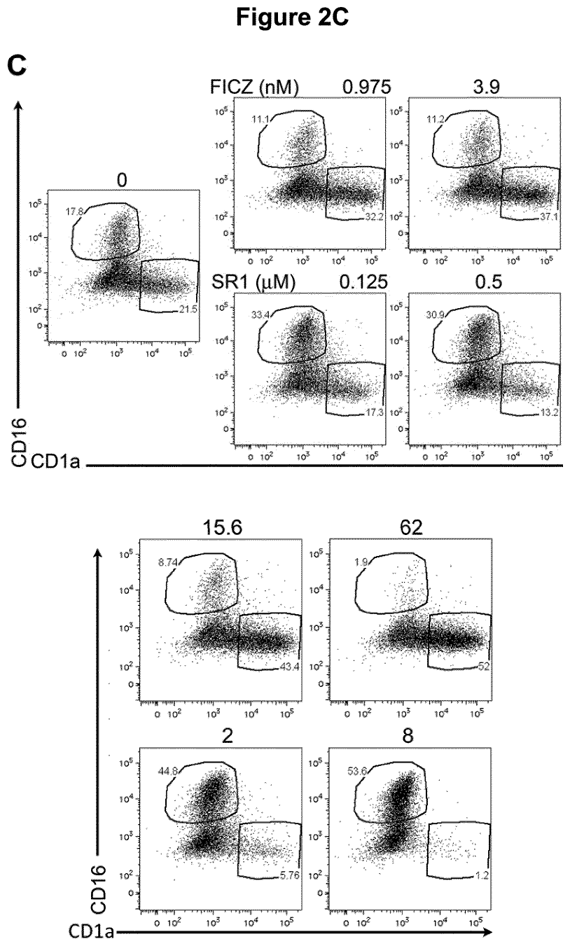

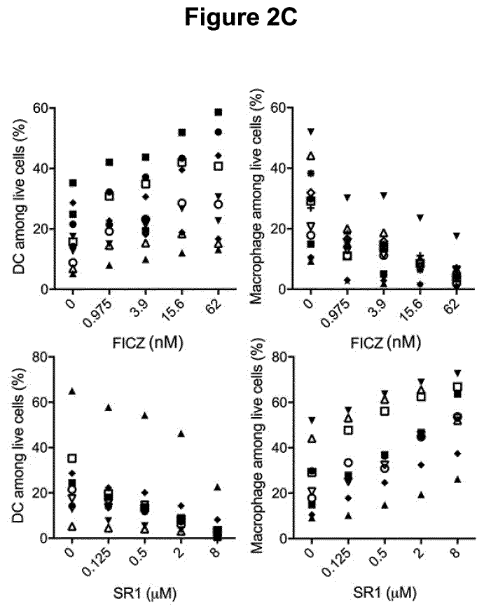

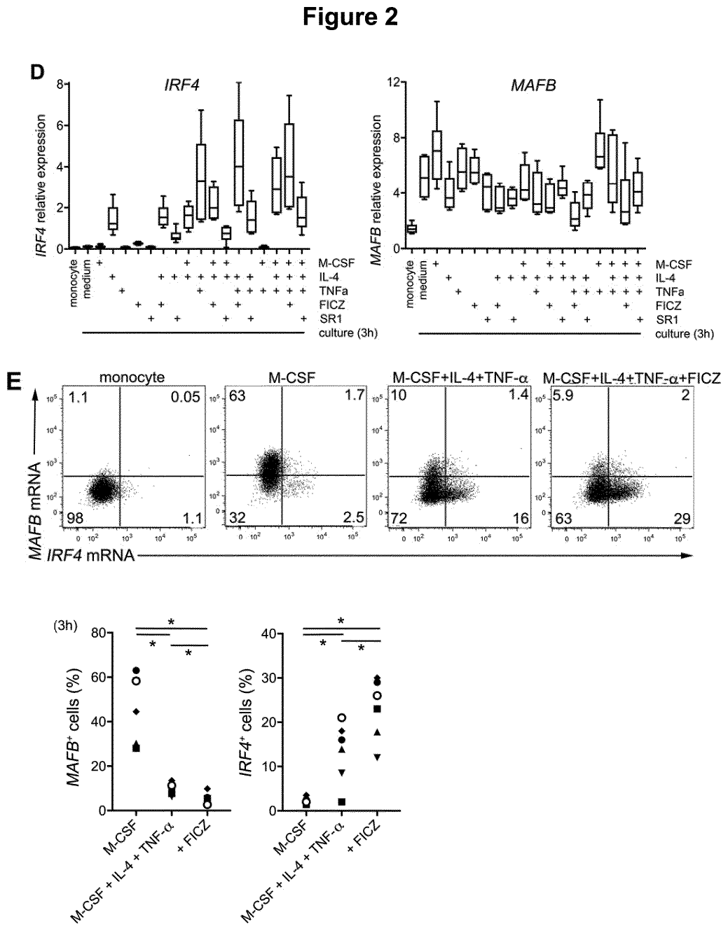

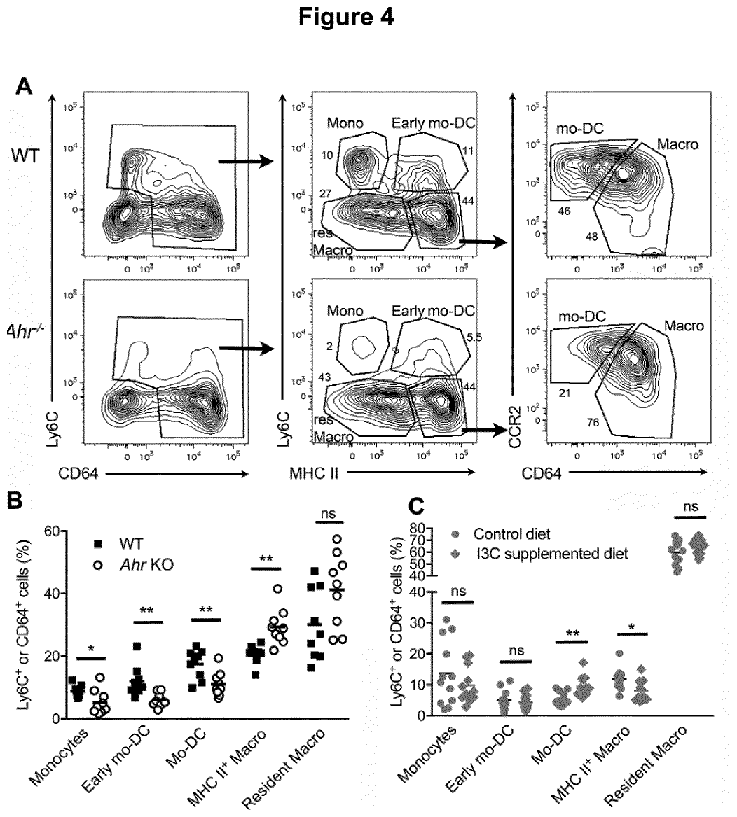

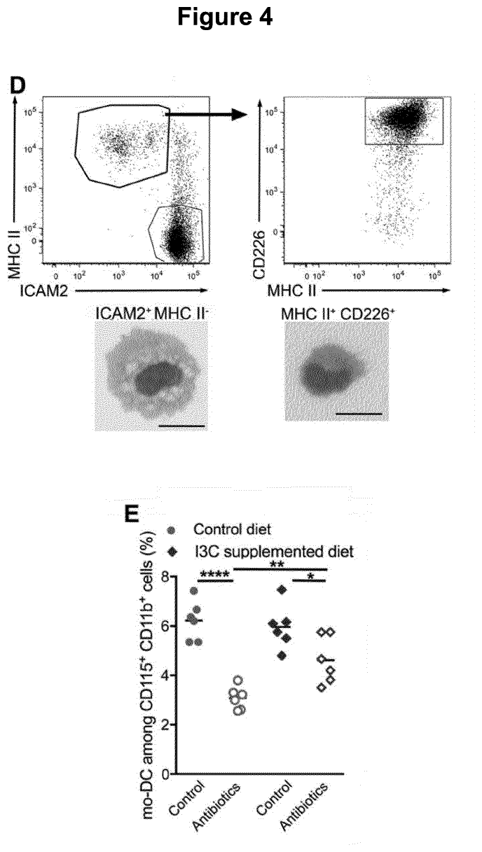

To address the role of AHR, we first inhibited its expression by targeted silencing using lentiviral vectors (FIG. 2A). AHR silencing reduced mo-DC differentiation while slightly increasing mo-Mac (FIG. 4B). Because culture medium contains small amounts of AHR ligands (Veldhoen et al., 2009) and AHR silencing was incomplete (FIG. 2A), we sought to confirm these results using a different approach. We cultured monocytes in the presence of various doses of a natural AHR agonist (6-Formylindolo(3,2-b)carbazole, FICZ) or an AHR inhibitor (stemregenin-1, SR1) and assessed mo-DC and mo-Mac differentiation. AHR activation by FICZ increased mo-DC while decreasing mo-Mac development (FIG. 2C). Conversely, AHR inhibition by SR1 increased mo-Mac while decreasing mo-DC proportions (FIG. 2C). Of note, the phenotype of FICZ-treated mo-DC or SR1-treated mo-Mac was similar to that of untreated cells, while the phenotype of CD1a⁻CD16⁻ cells remained unchanged. These results suggest that AHR is a molecular switch for mo-DC versus mo-Mac differentiation.

To decipher how AHR shapes monocyte fate, we assessed IRF4 and MAFB expression in monocytes after 3 h of culture with various combinations of M-CSF, IL-4, TNF-α, SR1 and FICZ (FIG. 2D). We also analyzed the expression of CYP1A1, a known direct target of AHR (Stockinger et al., 2014), as a control for AHR activation. IL-4 induced IRF4 expression, as previously reported (Lehtonen et al., 2005). This induction was inhibited in the presence of SR1, indicating that IRF4 expression is dependent on AHR signaling, presumably in response to small amounts of AHR ligand from the culture medium. However, IRF4 was not induced in the presence of FICZ alone. The expression of IL-4-induced IRF4 was further increased in the presence of TNF-α and with FICZ. By contrast, MAFB expression was induced by culture medium alone, and further increased by M-CSF (FIG. 2D). AHR signaling had no significant impact on MAFB expression at this time point. To address whether quantities of mRNA expressed per cell or proportions of expressing cells were regulated by these signals, we analyzed the expression of IRF4 and MAFB mRNA at the single-cell level using fluorescent in situ hybridization coupled to flow cytometry (FIG. 2E). After 3 h of culture, monocytes upregulated MAFB in response to M-CSF, but in the presence of IL-4 and TNF-α, the proportion of MAFB-expressing monocytes was dramatically reduced while a distinct population of IRF4-expressing monocytes appeared, which was further increased in the presence of FICZ. These results show that external signals polarize monocytes towards mo-DC versus mo-Mac differentiation. In line with this, increasing concentrations in the culture of IL-4 or TNF-α increased the proportion of mo-DC while reducing that of mo-Mac. In addition, the effect of FICZ on the culture outcome was strongly diminished in the absence of TNF-α. These results indicate that IL-4, TNF-α and AHR signaling synergize for IRF4 induction and mo-DC differentiation. Collectively, these results suggest the existence of a default differentiation pathway into mo-Mac, which can be switched to mo-DC differentiation in response to IL-4, TNF-α and AHR ligands.

### AHR Acts Through BLIMP-1

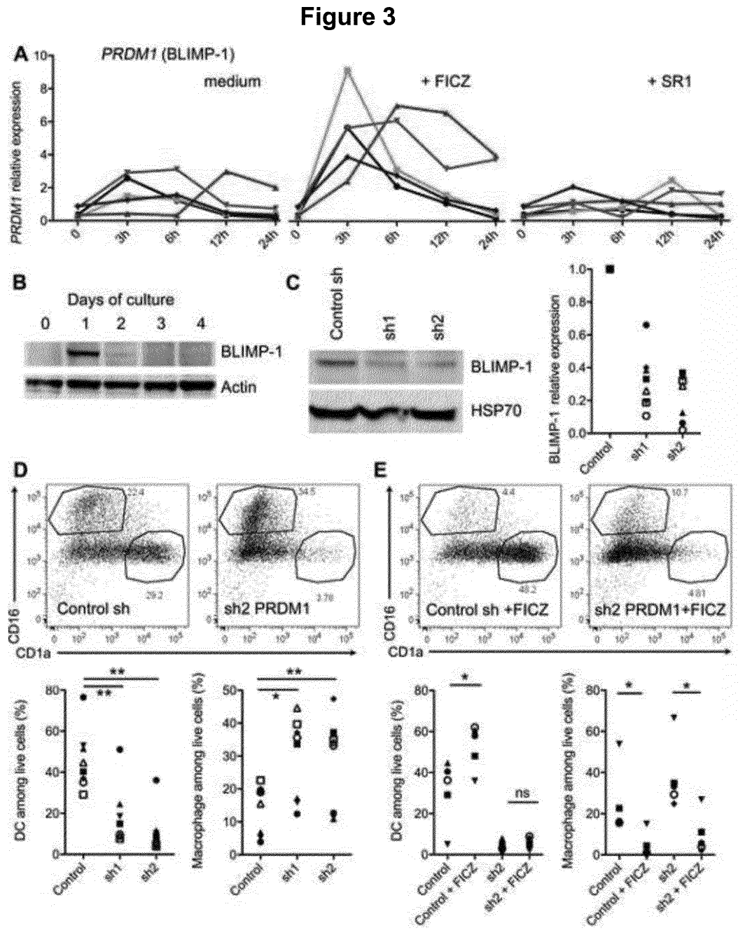

AHR activation triggers an autoregulatory feedback loop that restricts AHR signaling to a short timeframe (Stockinger et al., 2014). Therefore, we hypothesized that the effect of AHR activation on monocyte differentiation may be mediated by additional molecular regulators. In our differential transcriptomic analysis, we looked for transcription factors that could be induced by AHR activation based on literature. We identified PRDM1 (encoding BLIMP-1) as a candidate. Studies on cell lines have suggested that PRDM1 is a target of AHR (De Abrew et al., 2010; Ikuta et al., 2010). To analyze whether PRDM1 was induced by AHR signaling in monocytes, we measured PRDM1 expression during the early stages of monocyte culture in the presence or absence of FICZ or SR1 (FIG. 3A). We found that PRDM1 was rapidly induced upon AHR activation within 3 hours, suggesting that PRDM1 is a target of AHR. At the protein level, BLIMP-1 expression peaked during the first 24 h of the culture then decreased (FIG. 3B). To address whether PRDM1 was involved in monocyte differentiation, we silenced PRDM1 expression using shRNA (FIG. 3C). PRDM1 silencing significantly decreased mo-DC differentiation, while increasing the proportion of mo-Mac (FIG. 3D). To confirm that the effect of AHR was mediated by PRDM1, we silenced its expression in monocytes cultured in the presence or absence of FICZ (FIG. 3E). PRDM1 silencing abolished the promotion of mo-DC differentiation by FICZ, while the proportion of mo-Mac was not fully restored to that observed in control cells in the absence of FICZ. These results suggest that BLIMP-1 is essential for AHR-induced mo-DC differentiation.

### AHR is Involved in mo-DC Differentiation In Vivo in the Mouse

To address the physiological relevance of our findings, we analyzed the role of AhR in mo-DC and mo-Mac differentiation in vivo in the mouse. In the steady-state dermis, mo-DC and mo-Mac continuously differentiate in situ from monocytes recruited to the skin (Tamoutounour et al., 2013). Five populations of macrophages and monocyte-related cells have been described in mouse skin: dermal monocytes, mo-DC at an early stage of differentiation, fully differentiated mo-DC, MHC class II⁺ macrophages (which contain a majority of mo-Mac) and resident MHC class II⁻ macrophages (Tamoutounour et al., 2013). Transcriptomic analysis showed that AhR and Irf4 were more expressed in mo-DC than in macrophages, while MafB was more expressed in macrophages (GEO accession code GSE49358), consistent with our findings in human monocyte-derived cells. In AhR-deficient mice, the proportion of skin mo-DC was decreased as compared to wild type (WT) littermates, while the proportion of MHC class II⁺ macrophages were increased (FIG. 4A-B). Proportions of other skin DC subsets and of monocyte subsets in the spleen were similar between AhR-deficient and WT mice. To confirm these findings, we increased AhR ligand availability in vivo by feeding mice with an experimental diet enriched for indole-3-carbinole (I3C), which is converted into an AhR ligand by stomach acids (Bjeldanes et al., 1991). In mice fed with IC-supplemented diet, mo-DC were significantly increased and MHC class II⁺ macrophages were decreased in the skin compared to mice fed with a control diet (FIG. 4C). These results show that AhR is involved in the in vivo differentiation of dermal mo-DC.

Recently, Irf4 has been shown in the mouse to be involved in the differentiation of a population of peritoneal and pleural MHC II⁺CD226⁺ monocyte-derived cells, proposed to be mo-Mac (Kim et al., 2016). However, our results identified CD226 as a marker of human mo-DC, both in vitro and in ascites (FIG. 1D). To determine the identity of mouse MHC II⁺CD226⁺ monocyte-derived cells, we isolated them from the peritoneal lavage of C57BL/6 mice, and compared their morphology to that of ICAM2⁺ peritoneal macrophages (FIG. 4D). MHC II⁺CD226⁺ cells displayed a typical DC morphology, distinct from that of bona fide ICAM2⁺ macrophages. Consistent with this, MHC II⁺CD226⁺ cells did not express the macrophage marker MerTK and CD226 was highly expressed by dermal mo-DC, but not by dermal macrophages. These results identify Irf4-dependent MHC II⁺CD226⁺ cells as mo-DC. As previously reported (Kim et al., 2016), this population of peritoneal mo-DC is decreased upon antibiotics treatment (FIG. 4E). Antibiotics induce the loss of intestinal bacteria species that are a major source of endogenous AhR ligand (Zelante et al., 2013). To address whether the decrease of peritoneal mo-DC upon antibiotics treatment was AhR-dependent, we fed antibiotics-treated mice with a I3C-supplemented or control diet, and analyzed cells from the peritoneal lavage (FIG. 3E). Supplementation in AhR ligand restored mo-DC differentiation in antibiotics-treated mice almost up to normal proportions.

Collectively, these results show that AhR activation in response to environmental stimuli has a key role in driving monocyte fate towards mo-DC in vivo.

### AHR Activation Correlates With the Presence of mo-DC in Leprosy Lesions

Finally, to put these findings in the context of human disease, we assessed AHR signaling and monocyte-derived cells presence in leprosy lesions, that contain granulomas in which monocytes are constantly recruited (Russell et al., 2009; Schreiber and Sandor, 2010). We analyzed published micro-array data from lepromatous (L-lep) and tuberculoid (T-lep) leprosy lesions ((Montoya et al., 2009), GEO accession code GSE17763). Patients with L-lep lesions display poor immunological responses against Mycobacterium leprae, the causative agent of leprosy, while patients with T-lep lesions have strong anti-M. leprae T cell responses (Montoya et al., 2009). We first defined “AHR agonist” and “AHR antagonist” gene signatures based on publicly available transcriptomic data (Di Meglio et al., 2014). To assess whether AHR signaling was active in leprosy lesions, we performed Gene Set Enrichment Analysis (GSEA). We found that the “AHR agonist” signature was enriched in T-lep lesions, while the “AHR antagonist” signature was enriched in L-lep lesions. Consistent with this, AHRR and CYP1A1, which are upregulated upon AHR activation (Stockinger et al., 2014), were highly expressed in T-lep lesions while IFIT1, which is down-regulated by AHR (Di Meglio et al., 2014), was more expressed in L-lep lesions. These results suggest that the AHR pathway was preferentially activated in T-lep lesions. To address the presence of mo-Mac and mo-DC, we performed GSEA using our gene signatures. The mo-DC signature was enriched in T-lep lesions, while the mo-Mac signature was enriched in L-lep lesions. By contrast, gene signatures of human Langerhans cells, skin CD1c⁺ DC or dermal macrophages (from (Carpentier et al., 2016)) were not enriched in either dataset. Consistent with these results, MERTK, CD163 and FCGR3A (encoding CD16) were more expressed in L-lep lesions and CD1A, CD1B and FCER1A were more expressed in T-lep lesions. These results suggest that mo-DC are more abundant in T-lep lesions while mo-Mac are more numerous in L-lep lesions. This is in line with previous work showing the absence of CD1a⁺CD1b⁺CD1c⁺DC in L-lep lesions (Sieling et al., 1999) and the increased presence of CD163⁺ macrophages in L-lep lesions (Montoya et al., 2009). Collectively, these results show that AHR activation correlates with the selective presence of mo-DC in L-lep lesions.

### Discussion

In this work, we have identified transcription factors involved in the differentiation of monocytes either into mo-Mac (MAFB) or into mo-DC (IRF4, AHR, BLIMP-1). These results show that mo-DC and mo-Mac do not represent different states of polarized monocytes, but rather are distinct lineages controlled by two different sets of molecular regulators. Several studies have evidenced that monocyte differentiation into mo-DC or mo-Mac is context-dependent (Bain et al., 2013; Zigmond et al., 2012). Here we identified micro-environmental cues that shape monocyte fate. Our results suggest that, in the presence of M-CSF, monocytes differentiate into mo-Mac by default, unless they are exposed to certain cytokines (IL-4 and TNF-α) in conjunction with AHR ligands, promoting mo-DC differentiation.

Our conclusions are primarily based on the use of an in vitro human monocyte differentiation model, but are reinforced by the validation of our main finding in vivo in a mouse model and the correlation between AHR activation and mo-DC presence in leprosy lesions. In addition, BLIMP-1 and IRF4 were identified in both mouse and human as preferentially expressed in intestinal CD103⁺CD11b⁺ DC (a proposed mo-DC population), and mice deficient for Blimp-1 in DC displayed a strongly reduced population of CD103⁺CD11b⁺ DC in the intestine (Watchmaker et al., 2014), further supporting the physiological relevance of our findings.

It has been suggested that mouse monocytes can be separated into two subpopulations that are pre-committed to become mo-Mac in response to pathogens or mo-DC in response to GM-CSF (Menezes et al., 2016). Using two different datasets of single-cell RNA-seq, we could not identify distinct subpopulations of mo-DC and mo-Mac precursors within human CD14⁺ monocytes. This is consistent with a recent single-cell RNA-seq analysis showing that mouse Ly6C⁺ and Ly6C⁻ monocytes are not heterogeneous at the transcriptomic level (Mildner et al., 2017). Our results show that all monocyte subsets express a partial mo-Mac transcriptomic signature, but cues from the micro-environment can drive monocyte fate towards mo-DC or mo-Mac. One hypothesis to explain how the same signals can induce different outcomes within a transcriptionally homogeneous population could be the stochastic heterogeneity in chromatin accessibility (Buenrostro et al., 2015).

MafB is highly expressed by all mouse macrophage populations except for lung macrophages (Gautier et al., 2012). Based on in vitro over-expression in myeloid progenitor cells, MafB has been proposed to induce macrophage differentiation (Bakri et al., 2005; Kelly et al., 2000). However, subsequent work showed that MafB is dispensable both in vivo and in vitro for murine macrophage differentiation from fetal progenitors (Aziz et al., 2006), suggesting that MafB is not essential for the initial stages of differentiation of embryonic-derived macrophages. MafB is rather involved in their terminal differentiation by repressing self-renewal genes (Aziz et al., 2009). Whether MafB is important for the differentiation of mouse macrophages in an inflammatory setting remains to be addressed.

Irf4 is preferentially expressed by mouse CD11b⁺ DC. Whether it is required for their development, or rather their migration and survival, remains unclear (Murphy et al., 2015). We show that IRF4 was essential for human mo-DC differentiation, and its expression in human monocytes was induced by IL-4 in an AHR-dependent way. This is consistent with previous work showing IRF4 expression upon culture with IL-4 in human and mouse monocytes (Briseno et al., 2016; Lehtonen et al., 2005). In addition, Irf4^{-/-} mouse monocytes cultured with GM-CSF and IL-4 fail to differentiate into mo-DC, but rather become mo-Mac (Briseno et al., 2016), supporting the idea of a default differentiation pathway into mo-Mac. We also showed that mouse Irf4-dependent peritoneal monocyte-derived cells, initially described as mo-Mac (Kim et al., 2016), actually correspond to mo-DC, based on their morphology and phenotype.

Previous evidence suggests a major role for AhR in the control of inflammatory responses, in particular through its action on T helper-17 (Th17) cell development and innate lymphoid cells homeostasis (Stockinger et al., 2014). Our work highlights an additional level of control by AhR ligands, by switching monocyte differentiation towards mo-DC, which are major producers of IL-23 and inducers of Th17 cells (Segura et al., 2013). Natural AhR ligands are derived from dietary intake (Bjeldanes et al., 1991) or produced through tryptophan catabolism at mucosal barriers (Fritsche et al., 2007; Zelante et al., 2013). AhR ligands can circulate throughout the body as evidenced by the regulation of astrocyte activity by microbiota-derived AhR ligands (Zelante et al., 2013), or the presence in milk of AhR ligands derived from the maternal microbiota (Gomez de Aguero et al., 2016).

Several studies have evidenced a deleterious role of monocyte-derived cells in pathological situations. mo-DC induce pathogenic T cells that mediate tissue damage in mice models of autoimmune or inflammatory diseases such as experimental autoimmune encephalomyelitis (Croxford et al., 2015) and colitis (Zigmond et al., 2012). Human “inflammatory” mo-DC likely contribute to the pathogenesis in Crohn's disease, rheumatoid arthritis and psoriasis through the secretion of high amounts of IL-23 and the induction of Th17 cells (Kamada et al., 2008; Segura et al., 2013; Zaba et al., 2009), two major players in the pathogenesis of these diseases. In tumors, mo-Mac play a central role in the suppression of anti-tumoral immune responses (Noy and Pollard, 2014). Monocyte-derived cells have therefore emerged in the past few years as attractive therapeutic targets (Getts et al., 2014; Leuschner et al., 2011). By enabling a better understanding of the molecular ontogeny of human monocyte-derived cells, our results should provide new opportunities for the therapeutic manipulation of monocyte differentiation.

The publication Goudot C, Coillard A, Villani A C, Gueguen P, Cros A, Sarkizova S, Tang-Huau T L, Bohec M, Baulande S, Hacohen N, Amigorena S, Segura E (2017). Aryl hydrocarbon receptor controls monocyte differentiation into dendritic cells versus macrophages. Immunity. 47(3): 582-596, is incorporated by reference to the present application. This publication includes the results of example 1. Using an in vitro culture model yielding human mo-DC and mo-Mac closely resembling those found in vivo in ascites, the inventors showed that IRF4 and MAFB were critical regulators of monocyte differentiation into mo-DC and mo-Mac, respectively. Activation of the aryl hydrocarbon receptor (AHR) promoted mo-DC differentiation through the induction of BLIMP-1, while impairing differentiation into mo-Mac. AhR deficiency also impaired the in vivo differentiation of mouse mo-DC. Finally, AHR activation correlated with mo-DC infiltration in leprosy lesions. These results establish that mo-DC and mo-Mac are controlled by distinct transcription factors, and show that AHR acts as a molecular switch for monocyte fate specification in response to micro-environmental factors.

### Example 2: AhR Agonist Improves the Efficacy of Anti-PD1 Treatment in Tumor-Bearing Mice

### Material and Methods

### Mice

C57BL/6 female mice were obtained from Charles River Janvier and maintained under specific pathogen-free conditions at the animal facility of Institut Curie in accordance with institutional guidelines. C57BL/6 mice were maintained on a purified diet (AIN-93M, Safe diets) supplemented or not with 200 p.p.m. indole-3-carbinol (Sigma) for 3 weeks, starting when the mice were 3 weeks-old. 6 week-old mice used for tumor experiments.

### Cells

B16.F10 OVA-expressing cells or MCA.101 OVA-expressing cells (Zeelenberg et al., 2008) were grown in RPMI-1640 containing 10% heat-inactivated FBS (Biowest), 100 IU/ml penicillin, 100 μg/ml streptomycin, 2 mM GlutaMAX, and 50 μM β-mercaptoethanol (all from Thermo Fisher Scientific).

### Tumor Growth Experiments

Mice were injected subcutaneously in the flank with 0.5 10⁶ B16.F10-OVA melanoma cells or 0.5 10⁶ MCA.101-OVA cells. Tumor growth was measured twice a week and was followed until the tumor became necrotic or until the size reached 1,500 mm³. Mice were treated, or not, with anti-PD1 (Bio X cell) starting when the tumor was palpable for B16.F10-OVA or starting when the tumor was 100-200 mm³ for MCA.101-OVA. Treatment consisted of intra-peritoneal injections of 200 μg of each antibody, delivered at day 7, day 10 and day 13 for B16.F10-OVA, or day 7 and day 14 for MCA.101-OVA. Control treatment consisted of intra-peritoneal injections of the same volume of PBS.

### Results

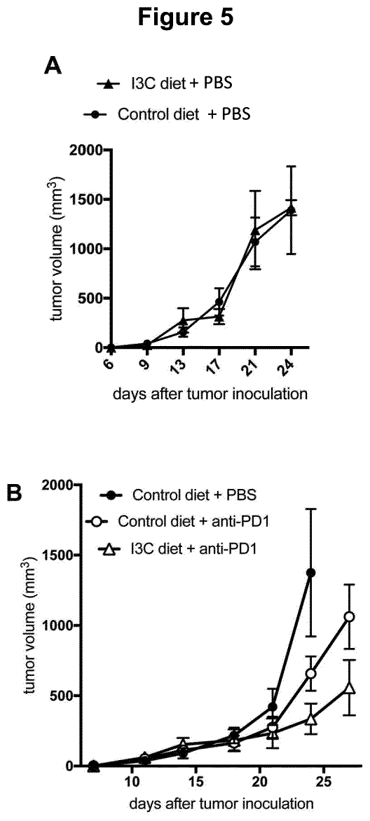

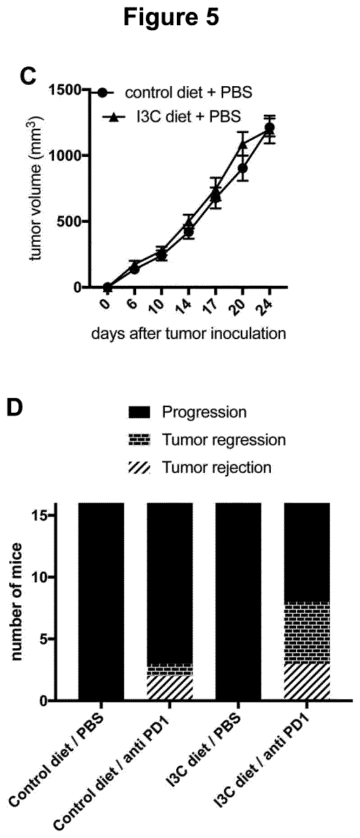

To address whether increasing the availability of Aryl Hydrocarbon Receptor (AhR) ligands improves spontaneous anti-tumoral responses, we inoculated B16.F10-OVA melanoma cells to mice fed with a control diet or supplemented with indole-3-carbinol (I3C), which is cleaved into AhR ligands by stomach acids (Bjeldanes et al., 1991). Tumor growth in mice fed with I3C-supplemented was similar to that of the control group (n=12 mice) (FIG. 5A).

To address whether increasing the availability of AhR ligands improves the efficacy of anti-checkpoint therapy, we treated mice with anti-PD1 3 times (at day 7, day 10 and day 13 post-inoculation of tumor cells). Tumor growth in mice fed with I3C-supplemented and treated with anti-PD1 therapy was significantly delayed compared to that of untreated mice and mice treated with anti-PD1 therapy and fed with the control diel (n=8 mice) (FIG. 5B).

To confirm these results, we repeated this experiment with a second tumor model, MCA.101-OVA cells. We inoculated MCA.101-OVA cells to mice fed with a control diet or supplemented with I3C. Tumor growth in mice fed with I3C-supplemented diet was similar to the control group (n=16 mice) (FIG. 5C).

To address whether increasing the availability of AhR ligands improves the efficacy of anti-PD1 therapy, we treated mice with anti-PD1 twice (at day 7 and day 14 post-inoculation of tumor cells). Tumor volume at end point was significantly reduced in mice fed with I3C-supplemented diet and treated with anti-PD1 therapy compared to that of mice treated with anti-PD1 therapy and fed with the control diet (n=16 mice) (FIG. 5D). In addition, response to treatment in mice fed with I3C-supplemented diet was significantly improved compared to that of mice fed with the control diet, with 50% of mice responding to anti-PD1 treatment (regression or complete rejection) in the I3C diet group, versus 18.75% of mice responding in the control group (n=16 mice) (FIG. 5D).

### REFERENCES

Aziz, A., Soucie, E., Sarrazin, S., and Sieweke, M. H. (2009). MafB/c-Maf deficiency enables self-renewal of differentiated functional macrophages. Science 326, 867-871.

Aziz, A., Vanhille, L., Mohideen, P., Kelly, L. M., Otto, C., Bakri, Y., Mossadegh, N., Sarrazin, S., and Sieweke, M. H. (2006). Development of macrophages with altered actin organization in the absence of MafB. Mol Cell Biol 26, 6808-6818.

Bain, C. C., Bravo-Blas, A., Scott, C. L., Gomez Perdiguero, E., Geissmann, F., Henri, S., Malissen, B., Osborne, L. C., Artis, D., and Mowat, A. M. (2014). Constant replenishment from circulating monocytes maintains the macrophage pool in the intestine of adult mice. Nat Immunol 15, 929-937.

Bain, C. C., Scott, C. L., Uronen-Hansson, H., Gudjonsson, S., Jansson, O., Grip, O., Guilliams, M., Malissen, B., Agace, W. W., and Mowat, A. M. (2013). Resident and pro-inflammatory macrophages in the colon represent alternative context-dependent fates of the same Ly6C(hi) monocyte precursors. Mucosal immunology 6, 498-510.

Bakri, Y., Sarrazin, S., Mayer, U. P., Tillmanns, S., Nerlov, C., Boned, A., and Sieweke, M. H. (2005). Balance of MafB and PU.1 specifies alternative macrophage or dendritic cell fate. Blood 105, 2707-2716.

Bjeldanes, L. F., Kim, J. Y., Grose, K. R., Bartholomew, J. C., and Bradfield, C. A. (1991). Aromatic hydrocarbon responsiveness-receptor agonists generated from indole-3-carbinol in vitro and in vivo: comparisons with 2,3,7,8-tetrachlorodibenzo-p-dioxin. Proc Natl Acad Sci U S A 88, 9543-9547.

Breton, G., Zheng, S., Valieris, R., Tojal da Silva, I., Satija, R., and Nussenzweig, M. C. (2016). Human dendritic cells (DCs) are derived from distinct circulating precursors that are precommitted to become CD1c+ or CD141+ DCs. J Exp Med.

Briseno, C. G., Haldar, M., Kretzer, N. M., Wu, X., Theisen, D. J., Kc, W., Durai, V., Grajales-Reyes, G. E., Iwata, A., Bagadia, P., et al. (2016). Distinct Transcriptional Programs Control Cross-Priming in Classical and Monocyte-Derived Dendritic Cells. Cell Rep 15, 2462-2474.

Buenrostro, J. D., Wu, B., Litzenburger, U. M., Ruff, D., Gonzales, M. L., Snyder, M. P., Chang, H. Y., and Greenleaf, W. J. (2015). Single-cell chromatin accessibility reveals principles of regulatory variation. Nature 523, 486-490.

Carpentier, S., Vu Manh, T. P., Chelbi, R., Henri, S., Malissen, B., Haniffa, M., Ginhoux, F., and Dalod, M. (2016). Comparative genomics analysis of mononuclear phagocyte subsets confirms homology between lymphoid tissue-resident and dermal XCR1(+) DCs in mouse and human and distinguishes them from Langerhans cells. Journal of immunological methods 432, 35-49.

Carvalho, B. S., and Irizarry, R. A. (2010). A framework for oligonucleotide microarray preprocessing. Bioinformatics 26, 2363-2367.

Chessel, D., Dufour, A. B., and Thioulouse, J. (2004). The ade4 package-I-One-table methods. R News 4.

Croxford, A. L., Lanzinger, M., Hartmann, F. J., Schreiner, B., Mair, F., Pelczar, P., Clausen, B. E., Jung, S., Greter, M., and Becher, B. (2015). The Cytokine GM-CSF Drives the Inflammatory Signature of CCR2 Monocytes and Licenses Autoimmunity. Immunity.

Davies, L. C., Rosas, M., Jenkins, S. J., Liao, C. T., Scurr, M. J., Brombacher, F., Fraser, D. J., Allen, J. E., Jones, S. A., and Taylor, P. R. (2013). Distinct bone marrow-derived and tissue-resident macrophage lineages proliferate at key stages during inflammation. Nature communications 4, 1886.

De Abrew, K. N., Kaminski, N. E., and Thomas, R. S. (2010). An integrated genomic analysis of aryl hydrocarbon receptor-mediated inhibition of B-cell differentiation. Toxicol Sci 118, 454-469.

Di Meglio, P., Duarte, J. H., Ahlfors, H., Owens, N. D., Li, Y., Villanova, F., Tosi, I., Hirota, K., Nestle, F. O., Mrowietz, U., et al. (2014). Activation of the aryl hydrocarbon receptor dampens the severity of inflammatory skin conditions. Immunity 40, 989-1001.

Eguiluz-Gracia, I., Bosco, A., Dollner, R., Melum, G. R., Lexberg, M. H., Jones, A. C., Dheyauldeen, S. A., Holt, P. G., Baekkevold, E. S., and Jahnsen, F. L. (2016). Rapid recruitment of CD14(+) monocytes in experimentally induced allergic rhinitis in human subjects. J Allergy Clin Immunol 137, 1872-1881 e1812.

Fernandez-Salguero, P., Pineau, T., Hilbert, D. M., McPhail, T., Lee, S. S., Kimura, S., Nebert, D. W., Rudikoff, S., Ward, J. M., and Gonzalez, F. J. (1995). Immune system impairment and hepatic fibrosis in mice lacking the dioxin-binding Ah receptor. Science 268, 722-726.

Fritsche, E., Schafer, C., Calles, C., Bernsmann, T., Bernshausen, T., Wurm, M., Hubenthal, U., Cline, J. E., Hajimiragha, H., Schroeder, P., et al. (2007). Lightening up the UV response by identification of the arylhydrocarbon receptor as a cytoplasmatic target for ultraviolet B radiation. Proc Natl Acad Sci USA 104, 8851-8856.

Gautier, E. L., Shay, T., Miller, J., Greter, M., Jakubzick, C., Ivanov, S., Helft, J., Chow, A., Elpek, K. G., Gordonov, S., et al. (2012). Gene-expression profiles and transcriptional regulatory pathways that underlie the identity and diversity of mouse tissue macrophages. Nat Immunol 13, 1118-1128.

Getts, D. R., Terry, R. L., Getts, M. T., Deffrasnes, C., Muller, M., van Vreden, C., Ashhurst, T. M., Chami, B., McCarthy, D., Wu, H., et al. (2014). Therapeutic inflammatory monocyte modulation using immune-modifying microparticles. Science translational medicine 6, 219ra217.

Gomez de Aguero, M., Ganal-Vonarburg, S. C., Fuhrer, T., Rupp, S., Uchimura, Y., Li, H., Steinert, A., Heikenwalder, M., Hapfelmeier, S., Sauer, U., et al. (2016). The maternal microbiota drives early postnatal innate immune development. Science 351, 1296-1302.

Gosselin, D., Link, V. M., Romanoski, C. E., Fonseca, G. J., Eichenfield, D. Z., Spann, N. J., Stender, J. D., Chun, H. B., Garner, H., Geissmann, F., and Glass, C. K. (2014). Environment drives selection and function of enhancers controlling tissue-specific macrophage identities. Cell 159, 1327-1340.

Greter, M., Helft, J., Chow, A., Hashimoto, D., Mortha, A., Agudo-Cantero, J., Bogunovic, M., Gautier, E. L., Miller, J., Leboeuf, M., et al. (2012). GM-CSF Controls Nonlymphoid Tissue Dendritic Cell Homeostasis but Is Dispensable for the Differentiation of Inflammatory Dendritic Cells. Immunity 36, 1031-1046.

Grimm, M. C., Pullman, W. E., Bennett, G. M., Sullivan, P. J., Pavli, P., and Doe, W. F. (1995). Direct evidence of monocyte recruitment to inflammatory bowel disease mucosa. J Gastroenterol Hepatol 10, 387-395.

Guilliams, M., Ginhoux, F., Jakubzick, C., Naik, S. H., Onai, N., Schraml, B. U., Segura, E., Tussiwand, R., and Yona, S. (2014). Dendritic cells, monocytes and macrophages: a unified nomenclature based on ontogeny. Nat Rev Immunol 14, 571-578.

Haldar, M., Kohyama, M., So, A. Y., Kc, W., Wu, X., Briseno, C. G., Satpathy, A. T., Kretzer, N. M., Arase, H., Rajasekaran, N. S., et al. (2014). Heme-mediated SPI-C induction promotes monocyte differentiation into iron-recycling macrophages. Cell 156, 1223-1234.

Ikuta, T., Ohba, M., Zouboulis, C. C., Fujii-Kuriyama, Y., and Kawajiri, K. (2010). B lymphocyte-induced maturation protein 1 is a novel target gene of aryl hydrocarbon receptor. J Dermatol Sci 58, 211-216.

Jenner, W., Motwani, M., Veighey, K., Newson, J., Audzevich, T., Nicolaou, A., Murphy, S., Macallister, R., and Gilroy, D. W. (2014). Characterisation of leukocytes in a human skin blister model of acute inflammation and resolution. PLoS One 9, e89375.

Kamada, N., Hisamatsu, T., Okamoto, S., Chinen, H., Kobayashi, T., Sato, T., Sakuraba, A., Kitazume, M. T., Sugita, A., Koganei, K., et al. (2008). Unique CD14 intestinal macrophages contribute to the pathogenesis of Crohn disease via IL-23/IFN-gamma axis. J Clin Invest 118, 2269-2280.

Kauffmann, A., Gentleman, R., and Huber, W. (2009). arrayQualityMetrics—a bioconductor package for quality assessment of microarray data. Bioinformatics 25, 415-416.

Kelly, L. M., Englmeier, U., Lafon, I., Sieweke, M. H., and Graf, T. (2000). MafB is an inducer of monocytic differentiation. The EMBO journal 19, 1987-1997.

Kim, K. W., Williams, J. W., Wang, Y. T., Ivanov, S., Gilfillan, S., Colonna, M., Virgin, H. W., Gautier, E. L., and Randolph, G. J. (2016). MHC II+ resident peritoneal and pleural macrophages rely on IRF4 for development from circulating monocytes. J Exp Med 213, 1951-1959.

Lavin, Y., Winter, D., Blecher-Gonen, R., David, E., Keren-Shaul, H., Merad, M., Jung, S., and Amit, I. (2014). Tissue-resident macrophage enhancer landscapes are shaped by the local microenvironment. Cell 159, 1312-1326.

Lehtonen, A., Veckman, V., Nikula, T., Lahesmaa, R., Kinnunen, L., Matikainen, S., and Julkunen, I. (2005). Differential expression of IFN regulatory factor 4 gene in human monocyte-derived dendritic cells and macrophages. J Immunol 175, 6570-6579.

Leuschner, F., Dutta, P., Gorbatov, R., Novobrantseva, T. I., Donahoe, J. S., Courties, G., Lee, K. M., Kim, J. I., Markmann, J. F., Marinelli, B., et al. (2011). Therapeutic siRNA silencing in inflammatory monocytes in mice. Nat Biotechnol 29, 1005-1010.

McGovern, N., Schlitzer, A., Gunawan, M., Jardine, L., Shin, A., Poyner, E., Green, K., Dickinson, R., Wang, X. N., Low, D., et al. (2014). Human Dermal CD14(+) Cells Are a Transient Population of Monocyte-Derived Macrophages. Immunity 41, 465-477.

Menezes, S., Melandri, D., Anselmi, G., Perchet, T., Loschko, J., Dubrot, J., Patel, R., Gautier, E. L., Hugues, S., Longhi, M. P., et al. (2016). The Heterogeneity of Ly6Chi Monocytes Controls Their Differentiation into iNOS+ Macrophages or Monocyte-Derived Dendritic Cells. Immunity 45, 1205-1218.

Mildner, A., Schonheit, J., Giladi, A., David, E., Lara-Astiaso, D., Lorenzo-Vivas, E., Paul, F., Chappell-Maor, L., Priller, J., Leutz, A., et al. (2017). Genomic Characterization of Murine Monocytes Reveals C/EBPbeta Transcription Factor Dependence of Ly6C-Cells. Immunity 46, 849-862 e847.

Mildner, A., Yona, S., and Jung, S. (2013). A close encounter of the third kind: monocyte-derived cells. Advances in immunology 120, 69-103.

Montoya, D., Cruz, D., Teles, R. M., Lee, D. J., Ochoa, M. T., Krutzik, S. R., Chun, R., Schenk, M., Zhang, X., Ferguson, B. G., et al. (2009). Divergence of macrophage phagocytic and antimicrobial programs in leprosy. Cell Host Microbe 6, 343-353.

Murphy, T. L., Grajales-Reyes, G. E., Wu, X., Tussiwand, R., Briseno, C. G., Iwata, A., Kretzer, N. M., Durai, V., and Murphy, K. M. (2015). Transcriptional Control of Dendritic Cell Development. Annu Rev Immunol.

Noy, R., and Pollard, J. W. (2014). Tumor-associated macrophages: from mechanisms to therapy. Immunity 41, 49-61.

Okabe, Y., and Medzhitov, R. (2014). Tissue-specific signals control reversible program of localization and functional polarization of macrophages. Cell 157, 832-844.

Patel, A. A., Zhang, Y., Fullerton, J. N., Boelen, L., Rongvaux, A., Maini, A. A., Bigley, V., Flavell, R. A., Gilroy, D. W., Asquith, B., et al. (2017). The fate and lifespan of human monocyte subsets in steady state and systemic inflammation. J Exp Med.

Russell, D. G., Cardona, P. J., Kim, M. J., Allain, S., and Altare, F. (2009). Foamy macrophages and the progression of the human tuberculosis granuloma. Nat Immunol 10, 943-948.

Satija, R., Farrell, J. A., Gennert, D., Schier, A. F., and Regev, A. (2015). Spatial reconstruction of single-cell gene expression data. Nat Biotechnol 33, 495-502.

Schlitzer, A., Sivakamasundari, V., Chen, J., Sumatoh, H. R., Schreuder, J., Lum, J., Malleret, B., Zhang, S., Larbi, A., Zolezzi, F., et al. (2015). Identification of cDC1- and cDC2-committed DC progenitors reveals early lineage priming at the common DC progenitor stage in the bone marrow. Nat Immunol 16, 718-728.

Schreiber, H. A., and Sandor, M. (2010). The role of dendritic cells in mycobacterium-induced granulomas. Immunology letters 130, 26-31.

Segura, E., and Amigorena, S. (2013). Inflammatory dendritic cells in mice and humans. Trends Immunol 34, 440-445.

Segura, E., Touzot, M., Bohineust, A., Cappuccio, A., Chiocchia, G., Hosmalin, A., Dalod, M., Soumelis, V., and Amigorena, S. (2013). Human Inflammatory Dendritic Cells Induce Th17 Cell Differentiation. Immunity 38, 336-348.

Servant, N., Gravier, E., Gestraud, P., Laurent, C., Paccard, C., Biton, A., Brito, I., Mandel, J., Asselain, B., Barillot, E., and Hupe, P. (2010). EMA-A R package for Easy Microarray data analysis. BMC Res Notes 3, 277.

Sieling, P. A., Jullien, D., Dahlem, M., Tedder, T. F., Rea, T. H., Modlin, R. L., and Porcelli, S. A. (1999). CD1 expression by dendritic cells in human leprosy lesions: correlation with effective host immunity. J Immunol 162, 1851-1858.

Smyth, G. K. (2004). Linear models and empirical bayes methods for assessing differential expression in microarray experiments. Stat Appl Genet Mol Biol 3, Article3.

Stockinger, B., Di Meglio, P., Gialitakis, M., and Duarte, J. H. (2014). The aryl hydrocarbon receptor: multitasking in the immune system. Annu Rev Immunol 32, 403-432.

Tamoutounour, S., Guilliams, M., Montanana Sanchis, F., Liu, H., Terhorst, D., Malosse, C., Pollet, E., Ardouin, L., Luche, H., Sanchez, C., et al. (2013). Origins and functional specialization of macrophages and of conventional and monocyte-derived dendritic cells in mouse skin. Immunity 39, 925-938.

van der Maaten, L. (2013). Barnes-Hut-SNE. Proceedings of the International Conference on Learning Representations.

Veldhoen, M., Hirota, K., Christensen, J., O'Garra, A., and Stockinger, B. (2009). Natural agonists for aryl hydrocarbon receptor in culture medium are essential for optimal differentiation of Th17 T cells. J Exp Med 206, 43-49.

Villani, A. C., Satija, R., Reynolds, G., Sarkizova, S., Shekhar, K., Fletcher, J., Griesbeck, M., Butler, A., Zheng, S., Lazo, S., et al. (2017). Single-cell RNA-seq reveals new types of human blood dendritic cells, monocytes, and progenitors. Science 356.

Watchmaker, P. B., Lahl, K., Lee, M., Baumjohann, D., Morton, J., Kim, S. J., Zeng, R., Dent, A., Ansel, K. M., Diamond, B., et al. (2014). Comparative transcriptional and functional profiling defines conserved programs of intestinal DC differentiation in humans and mice. Nat Immunol 15, 98-108.

Wollenberg, A., Kraft, S., Hanau, D., and Bieber, T. (1996). Immunomorphological and ultrastructural characterization of Langerhans cells and a novel, inflammatory dendritic epidermal cell (IDEC) population in lesional skin of atopic eczema. J Invest Dermatol 106, 446-453.

Wong, K. L., Tai, J. J., Wong, W. C., Han, H., Sem, X., Yeap, W. H., Kourilsky, P., and Wong, S. C. (2011). Gene expression profiling reveals the defining features of the classical, intermediate, and nonclassical human monocyte subsets. Blood 118, e16-31.

Zaba, L. C., Fuentes-Duculan, J., Eungdamrong, N. J., Abello, M. V., Novitskaya, I., Pierson, K. C., Gonzalez, J., Krueger, J. G., and Lowes, M. A. (2009). Psoriasis is characterized by accumulation of immunostimulatory and Th1/Th17 cell-polarizing myeloid dendritic cells. J Invest Dermatol 129, 79-88.

Zeelenberg, I. S., M. Ostrowski, S. Krumeich, A. Bobrie, C. Jancic, A. Boissonnas, A. Delcayre, J. B. Le Pecq, B. Combadiere, S. Amigorena and C. Thery (2008). “Targeting tumor antigens to secreted membrane vesicles in vivo induces efficient antitumor immune responses.” Cancer Res 68(4): 1228-1235.

Zelante, T., Iannitti, R. G., Cunha, C., De Luca, A., Giovannini, G., Pieraccini, G., Zecchi, R., D'Angelo, C., Massi-Benedetti, C., Fallarino, F., et al. (2013). Tryptophan catabolites from microbiota engage aryl hydrocarbon receptor and balance mucosal reactivity via interleukin-22. Immunity 39, 372-385.

Zheng, G. X., Terry, J. M., Belgrader, P., Ryvkin, P., Bent, Z. W., Wilson, R., Ziraldo, S. B., Wheeler, T. D., McDermott, G. P., Zhu, J., et al. (2017). Massively parallel digital transcriptional profiling of single cells. Nature communications 8, 14049.

Zigmond, E., Varol, C., Farache, J., Elmaliah, E., Satpathy, A. T., Friedlander, G., Mack, M., Shpigel, N., Boneca, I. G., Murphy, K. M., et al. (2012). Ly6C(hi) Monocytes in the Inflamed Colon Give Rise to Proinflammatory Effector Cells and Migratory Antigen-Presenting Cells. Immunity 37, 1076-1090.

1. An AhR agonist for use in combination with at least one immune checkpoint modulator in the treatment of cancer.

2. An AhR agonist for use according to claim 1, wherein the AhR agonist is selected from the group comprising dietary indoles, dietary flavonoids, tryptophan metabolites and synthetic weak AhR agonists.

3. An AhR agonist for use according to claim 1, wherein the AhR agonist is a dietary indole or a derivative thereof, preferably the dietary indole or derivative thereof is an indole glucosinolate, preferably the dietary indole or derivative thereof is selected from the group comprising indole-3-carbinol, 3,3′-diindoylmethane, and indolo[3,2b]carbazole.

4. An AhR agonist for use according to claim 1, wherein the AhR agonist is a dietary flavonoid or derivative thereof, preferably the dietary flavonoid or derivative thereof is selected from the group comprising quercetin, galangin, daidzein, naringenin, baicalein, diosmin and diametin.

5. An AhR agonist for use according to claim 1, wherein the AhR agonist is a dietary indole or a dietary flavonoid, preferably the dietary indole or the dietary flavonoid is in the form of a natural product extract, preferably the AhR agonist is a dietary indole in the form of a cruciferous vegetable extract.

6. An AhR agonist for use according to claim 1, wherein the AhR agonist is a synthetic weak AhR agonist, preferably the synthetic weak AhR agonist is selected from the group comprising benzimidazole derivatives, such as omeprazole and lansoprazole, primaquine, leflutamide, VAF347 ([4-(3-chloro-phenyl)-pyrimidin-2-yl]-(4trifluoromethyl-phenyl)-amine), TSU-16 ((Z)-3-[(2,4-dimethylpyrrol-5-yl)methylidenyl]-2indolinone), synthetic flavonoids such as TMF (6,2′,4′-trimethoxyflavone) and MNF (3′-methoxy-4′nitroflavone), M50367 (ethyl 3-hydroxy-3-[2-(2-phenylethyl)benzoimidazol-4-yl]propanoate), M50354 (3-[2-(2-phenylethyl)benzoimidazole-4-yl]-3-hydroxypropanoic acid).

7. An AhR agonist for use according to claim 1, wherein the AhR agonist is a tryptophan metabolite, preferably the tryptophan metabolite is selected from the group comprising Kynurenic acid, Kynurenine, 6-formylindolo[3,2b]carbazole (FICZ) and Indoxyl sulfate.

8. An AhR agonist for use according to claim 1, wherein the AhR agonist is suitable for oral administration, preferably the AhR agonist is in the form of a medical food composition.

9. An AhR agonist for use in combination with at least one immune checkpoint regulator according to claim 1, wherein said at least one immune checkpoint modulator is an inhibitory immune checkpoint molecule and/or a stimulatory immune checkpoint agonist.

10. An AhR agonist for use in combination with at least one immune checkpoint regulator according to claim 9, wherein the inhibitory immune checkpoint molecule is selected from A2AR, B7-H3, B7-H4, BTLA, CTLA-4, CD277, IDO, KIR, PD-1, LAG-3, TIM-3 TIGIT, VISTA, CD96, CD112R, CD160, CD244 (or 2B4) DCIR (C-type lectin surface receptor), ILT3, ILT4 (Immunoglobulin-like transcript), CD31 (PECAM-1) (Ig-like R family), CD39, CD73, CD94/NKG2, GP49b (immunoglobulin superfamily), KLRG1, LAIR-1 (Leukocyte-associated immunoglobulin-like receptor 1) CD305 , PD-L1, PD-L2 and SIRPα, preferably, the inhibitory checkpoint molecule is selected from CTLA-4, PD1, PDL1 or a combination thereof.

11. An AhR agonist for use in combination with at least one immune checkpoint regulator according to claim 9, wherein the stimulatory immune checkpoint agonist is selected from CD27, CD40, OX40, GITR, ICOS, TNFRSF25, 41BB, HVEM, CD28, TMIGD2, CD226, 2B4 (CD244) and agonist CD48, B7-H6 Brandt (NK agonist), LIGHT (CD258, TNFSF14) and CD28H.

12. An AhR agonist for use in combination with at least one immune checkpoint modulator according to claim 1, wherein the immune checkpoint modulator is an antibody or a fusion protein.

13. An AhR agonist for use in combination with at least one immune checkpoint modulator according to claim 1, wherein the immune checkpoint modulator is an anti-PD-1 or an anti-PD-L1 antibody.

14. A product containing an AhR agonist and at least one immune checkpoint modulator as defined in claim 1, as a combined preparation for simultaneous, separate or sequential use in the treatment of cancer.

15. Method for treating a patient suffering from cancer, wherein said method comprises the combined administration of an effective amount of one or more AHR agonist and an effective amount of at least one immune checkpoint modulator.
